(19) United States
(12) Patent Application Publication
(10) Pub. No.: US 2025/0046464 A1
(43) Pub. Date: Feb. 6, 2025

(54) STATIC BALANCE ESTIMATION DEVICE, STATIC BALANCE ESTIMATION SYSTEM, STATIC BALANCE ESTIMATION METHOD, AND RECORDING MEDIUM

(71) Applicant: NEC Corporation, Minato-ku, Tokyo (JP)

(72) Inventors: Chenhui HUANG, Tokyo (JP); Fumiyuki NIHEY, Tokyo (JP); Zhenwei WANG, Tokyo (JP); Hiroshi KAJITANI, Tokyo (JP); Yoshitaka NOZAKI, Tokyo (JP); Kenichiro FUKUSHI, Tokyo (JP)

(73) Assignee: NEC Corporation, Minato-ku, Tokyo (JP)

(21) Appl. No.: 18/716,641

(22) PCT Filed: Dec. 27, 2021
(86) PCT No.: PCT/JP2021/048562

§ 371 Date: Jun. 5, 2024

(51) Int. Cl. G16H 50/30

(52) U.S. Cl. CPC G16H 50/30

(57) ABSTRACT

Provided is a static balance estimation device that includes a data acquisition unit that acquires feature amount data that include a feature amount to be used for estimating static balance of a user, the feature amount being extracted from a feature of a gait of the user, a storage unit that stores an estimation model that outputs a static balance index related to input of the feature amount data, an estimation unit that inputs the acquired feature amount data into the estimation model and estimates the static balance of the user according to the static balance index output by the estimation model, and an output unit that outputs information related to the estimated static balance of the user.

### TECHNICAL FIELD

The present disclosure relates to a static balance estimation device or the like that estimates static balance by using data regarding a gait.

### BACKGROUND ART

With growing interest in healthcare, services that provide information according to features (also referred to as gait) included in a gait pattern have attracted attention. For example, a technique for analyzing a gait based on sensor data measured by a sensor mounted in footwear such as shoes has been developed. In the time series data of the sensor data, a feature of a gait event (also referred to as a walking event) related to a physical condition appears.

PTL 1 discloses an estimation device that estimates the type of footwear using sensor data acquired from a sensor installed at the footwear. The device of PTL 1 extracts a characteristic gait feature amount in a gait with footwear using data acquired from a sensor installed at the footwear. The device of PTL 1 estimates the type of footwear based on the extracted gait feature amount.

PTL 2 discloses a system for monitoring a user's movement ability using evaluation based on clinical mobility. The system of PTL 2 has an inertial measurement device including a gyroscope and an accelerometer. The system of PTL 2 generates user inertial data indicating a user's movement ability based on evaluation according to clinical mobility using an inertial measurement device. The system of PTL 2 locally logs inertial data of a user in a mobile device. The system of PTL 2 determines the position and orientation of the mobile device in the evaluation period based on the clinical mobility by processing locally logged inertial data of the user in real time. The system of PTL 2 determines the user's body movement assessment related to the evaluation based on the clinical mobility using the position and orientation of the mobile device in the evaluation period based on the clinical mobility. The system of PTL 2 displays at least part of the body movement assessment to the user. PTL 2 exemplifies several tests as evaluation based on clinical mobility. For example, a time-up and go test, a chair rise test, a four-stage balance test, a gait analysis, a one leg standing position test, a sit-and-reach test, an arm curl test, posture stability, and the like are exemplified.

The one leg standing position test is one of tests for evaluating static balance and stability. The performance of the one leg standing position test is an important index for evaluating static balance and stability. In the one leg standing position test, the body operates to maintain stability from the pelvis to the lower limbs in order to control the wobble of the center of gravity of the front, back, left, and right. In the one leg standing position test, more motion control is performed on the coronal plane and the horizontal plane than on the sagittal plane.

NPL 1 reports the results of measuring the center of gravity oscillations of 33 healthy women in the one leg standing position with the eyes open and studying the relationship with main lower limb muscle strength and foot function. NPL 1 reports that the Musculus tibialis anterior on the standing leg side, the abductor hallucis muscle, the flexor digitorum Brevis muscle, the soleus muscle, the flexor hallucis Brevis muscle medial head, the Musculus quadriceps femoris, and the mesogluteus are related to posture retention in the one leg standing position. Specifically, NPL 1 reports a result suggesting that muscles related to the foot-gripping force, such as the Musculus tibialis anterior, the abductor hallucis muscle, the flexor digitorum Brevis muscle, the soleus muscle, and the flexor hallucis Brevis muscle medial head, are related to posture retention in the one leg standing position.

NPL 2 reports a relationship between balance and muscles according to age. NPL 2 reports that the muscles of the hip joint are more related to the balance than the knee joint and the ankle joint in the elderly. In particular, NPL 2 reports that a difference between the young and the elderly in the relationship between the muscles of the hip joint and the balance is remarkable in the one foot standing position test in the state where the eyes are closed.

NPL 3 reports the influence of aging and posture on holding a one leg standing position. NPL 3 reports that compared with in the posture of slightly raising the lower limb to such an extent that the lower limb does not touch the ground, in the posture of keeping the hip joint in the 90 degree bending position, the center of gravity velocity in the front-back direction, the inclination angle of the pelvis, and the lower limb muscle activity amount significantly increase in the elderly. NPL 3 reports that with respect to the center of gravity velocity in the one foot standing position, the main effect is observed in the muscle activity amount of the Musculus tibialis anterior, the Musculus rectus femoris, the Musculus biceps femoris, the mesogluteus, the Musculus tensor fasciae latae, the adductor, and the long fibular muscle.

### CITATION LIST

### Patent Literature

PTL 1: WO 2021/130907 A1
PTL 2: JP 2021-524075 A

### Non Patent Literature

NPL 1: Shin Murata, “Relationship between Body Sway of One-leg Standing with Vision and Foot Function in Healthy Female”, Physical Therapy Science, Vol. 19(3), pp. 245-249, 2004.
NPL 2: D. Wiksten et. al., “The relationship between muscle and balance performance as a function of age”, Isokinetics and Exercise Science, Vol. 6 (2), pp. 125-132, 1996.
NPL 3: Mariko Nambu, “Influence of aging and posture on holding a one leg standing position”, Department of Health Science, The Hokkaido University School of Medicine, Graduation Research Paper, 2013.

### SUMMARY OF INVENTION

### Technical Problem

The method of PTL 1 includes estimating the type of the footwear using the gait feature amount of the feature portion extracted from the data acquired from the sensor installed at the footwear. PTL 1 does not disclose estimating the static balance using the gait feature amount of the feature portion extracted from the data acquired from the sensor installed at the footwear.

PTL 2 exemplifies performing several tests in order to perform evaluation based on clinical mobility using inertial data measured by an inertial measurement device. In the method of PTL 2, it is necessary to actually perform some tests in order to perform evaluation based on clinical mobility.

As in NPLs 1 to 3, when the result of the one leg standing position test can be evaluated, the static balance can be evaluated. However, NPLs 1 to 3 does not disclose a method of evaluating static balance such as a one leg standing position test in daily life.

An object of the present disclosure is to provide a static balance estimation device and the like capable of appropriately estimating static balance indicating a knee state in daily life.

### Solution to Problem

A static balance estimation device according to an aspect of the present disclosure includes a data acquisition unit that acquires feature amount data that include a feature amount to be used for estimating static balance of a user, the feature amount being extracted from a feature of a gait of the user, a storage unit that stores an estimation model that outputs a static balance index related to input of the feature amount data, an estimation unit that inputs the acquired feature amount data into the estimation model and estimates the static balance of the user according to the static balance index output by the estimation model, and an output unit that outputs information related to the estimated static balance of the user.

A method of estimating static balance according to an aspect of the present disclosure includes acquiring feature amount data that include a feature amount to be used for estimating static balance of a user, the static balance being extracted from a feature of a gait of the user, inputting the acquired feature amount data to an estimation model that outputs a static balance index according to input of the feature amount data, estimating the static balance of the user according to the static balance index output from the estimation model, and outputting information related to the estimated static balance of the user.

A program according to an aspect of the present disclosure causes a computer to execute the steps of acquiring feature amount data that include a feature amount to be used for estimating static balance of a user, the static balance being extracted from a feature of a gait of the user, inputting the acquired feature amount data to an estimation model that outputs a static balance index according to input of the feature amount data, estimating the static balance of the user according to the static balance index output from the estimation model, and outputting information related to the estimated static balance of the user.

### Advantageous Effects of Invention

According to the present disclosure, it is possible to provide a static balance estimation device and the like capable of appropriately estimating static balance in daily life.

### BRIEF DESCRIPTION OF DRAWINGS

FIG. 1 is a block diagram illustrating an example of a configuration of a static balance estimation system according to a first example embodiment.

FIG. 2 is a block diagram illustrating an example of a configuration of a gait measurement device included in the static balance estimation system according to the first example embodiment.

FIG. 3 is a conceptual diagram illustrating an arrangement example of the gait measurement device according to the first example embodiment.

FIG. 4 is a conceptual diagram for describing an example of a relationship between a local coordinate system and a world coordinate system set in the gait measurement device according to the first example embodiment.

FIG. 5 is a conceptual diagram for describing a human body surface used in the description of the gait measurement device according to the first example embodiment.

FIG. 6 is a conceptual diagram for describing a gait cycle used in the description regarding the gait measurement device according to the first example embodiment.

FIG. 7 is a conceptual diagram for describing a gait parameter used in the description regarding the gait measurement device according to the first example embodiment.

FIG. 8 is a graph for describing an example of time series data of sensor data measured by the gait measurement device according to the first example embodiment.

FIG. 9 is a diagram for describing an example of normalization of gait waveform data extracted from time series data of sensor data measured by the gait measurement device according to the first example embodiment.

FIG. 10 is a conceptual diagram for describing an example of a gait phase cluster from which a feature amount data generation unit of the gait measurement device according to the first example embodiment extracts a feature amount.

FIG. 11 is a block diagram illustrating an example of a configuration of a static balance estimation device included in a static balance estimation system according to a first example embodiment.

FIG. 12 is a conceptual diagram for describing a one leg standing position test to be evaluated of the static balance estimation system according to the first example embodiment.

FIG. 13 is a table related to specific examples of feature amounts extracted by the gait measurement device included in the static balance estimation system according to the first example embodiment in order to estimate the one leg standing position time.

FIG. 14 is a graph illustrating a correlation between a feature amount F1 extracted by the gait measurement device included in the static balance estimation system according to the first example embodiment and a measured one leg standing position time.

FIG. 15 is a graph illustrating a correlation between a feature amount F2 extracted by the gait measurement device included in the static balance estimation system according to the first example embodiment and a measured one leg standing position time.

FIG. 16 is a graph illustrating a correlation between a feature amount F3 extracted by the gait measurement device included in the static balance estimation system according to the first example embodiment and a measured one leg standing position time.

FIG. 17 is a graph illustrating a correlation between a feature amount F4 extracted by the gait measurement device included in the static balance estimation system according to the first example embodiment and a measured one leg standing position time.

FIG. 18 is a graph illustrating a correlation between a feature amount F5 extracted by the gait measurement device included in the static balance estimation system according to the first example embodiment and a measured one leg standing position time.

FIG. 19 is a graph illustrating a correlation between a feature amount F6 extracted by the gait measurement device included in the static balance estimation system according to the first example embodiment and a measured one leg standing position time.

FIG. 20 is a graph illustrating a correlation between a feature amount F7 extracted by the gait measurement device included in the static balance estimation system according to the first example embodiment and a measured one leg standing position time.

FIG. 21 is a block diagram illustrating an example of estimation of a one leg standing position time (static balance index) by the static balance estimation device included in the static balance estimation system according to the first example embodiment.

FIG. 22 is a graph illustrating a correlation between an estimation value of a one leg standing position time estimated using an estimation model generated by machine learning with gender, age, height, weight, and gait speed as explanatory variables and a measurement value of the one leg standing position time.

FIG. 23 is a graph illustrating a correlation between an estimation value of the one leg standing position time estimated by the static balance estimation device included in the static balance estimation system according to the first example embodiment and a measurement value of the one leg standing position time.

FIG. 24 is a flowchart for describing an example of the operation of the gait measurement device included in the static balance estimation system according to the first example embodiment.

FIG. 25 is a flowchart for describing an example of the operation of the static balance estimation device included in the static balance estimation system according to the first example embodiment.

FIG. 26 is a conceptual diagram for describing an application example of the static balance estimation system according to the first example embodiment.

FIG. 27 is a block diagram illustrating an example of a configuration of a machine learning system according to a second example embodiment.

FIG. 28 is a block diagram illustrating an example of a configuration of a machine learning device included in a machine learning system according to the second example embodiment.

FIG. 29 is a conceptual diagram for describing an example of machine learning by a machine learning device included in a machine learning system according to the second example embodiment.

FIG. 30 is a block diagram illustrating an example of a configuration of a static balance estimation device according to a third example embodiment.

FIG. 31 is a block diagram illustrating an example of a hardware configuration that executes control and processing according to each example embodiment.

### EXAMPLE EMBODIMENT

Hereinafter, embodiments of the present invention will be described with reference to the drawings. However, the example embodiments described below have technically preferable limitations for carrying out the present invention, but the scope of the present invention is not limited to the following. In all the drawings used in the following description of the example embodiment, the same reference numerals are given to the same parts unless there is a particular reason. In the following example embodiments, repeated description of similar configurations and operations may be omitted.

### First Example Embodiment

First, a static balance estimation system according to a first example embodiment will be described with reference to the drawings. The static balance estimation system according to the present example embodiment measures sensor data related to motion of a foot according to a gait of a user. The static balance estimation system of the present example embodiment estimates the static balance of the user by using the measured sensor data. The sensor data is not limited to the sensor data regarding the motion of the foot, and may include a feature regarding the gait. For example, the sensor data may be sensor data including features related to a gait measured using motion capture, smart apparel, or the like.

In the present example embodiment, an example of estimating the result of the one leg standing position test as the static balance will be described. Specifically, in the present example embodiment, an example of estimating a result of a one leg standing position test (closed eye one leg standing position test) in a state where eyes are closed will be described. In the present example embodiment, the performance of the one leg standing position test is evaluated for a time (also referred to as a one foot standing position time) during which a state in which one leg is raised from the ground by 5 cm (centimeters) is maintained. The longer the one leg standing position time, the better the result of the one leg standing position test. The method of the present example embodiment can be applied to other than the closed eye one leg standing position test. For example, the method of the present example embodiment can also be applied to variations of a one leg standing position test (an open eye one leg standing position test) in a state where eyes are opened and other one leg standing position tests.

### (Configuration)

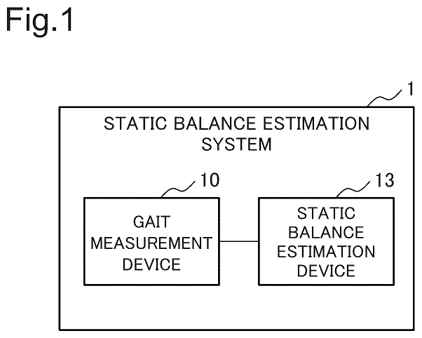

FIG. 1 is a block diagram illustrating an example of a configuration of a static balance estimation system 1 according to the present example embodiment. The static balance estimation system 1 includes a gait measurement device 10 and a static balance estimation device 13. In the present example embodiment, an example in which the gait measurement device 10 and the static balance estimation device 13 are configured as separate hardware will be described. For example, the gait measurement device 10 is installed in footwear or the like of a subject (user) whose static balance is to be estimated. For example, the function of the static balance estimation device 13 is installed in a mobile terminal carried by a subject (user). Hereinafter, configurations of the gait measurement device 10 and the static balance estimation device 13 will be individually described.

### [Gait Measurement Device]

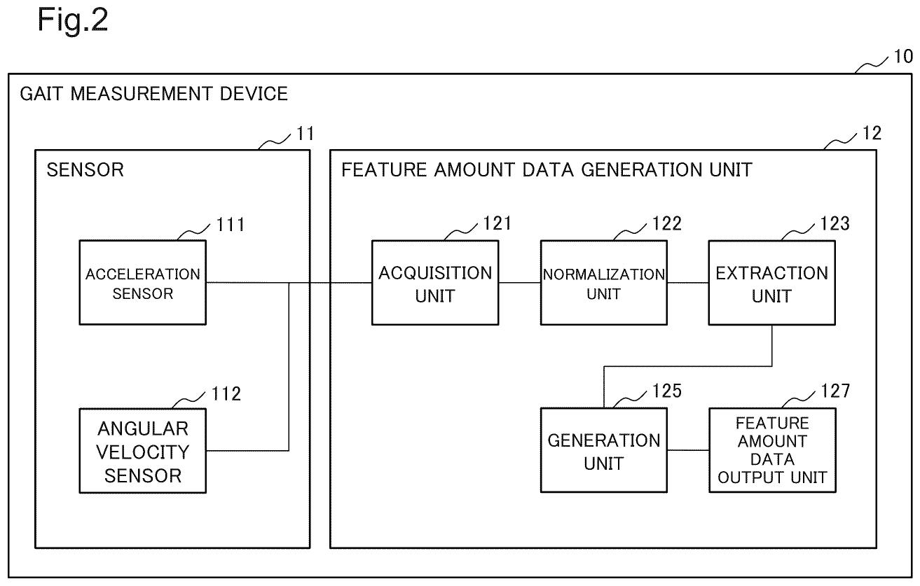

FIG. 2 is a block diagram illustrating an example of a configuration of the gait measurement device 10. The gait measurement device 10 includes a sensor 11 and a feature amount data generation unit 12. In the present example embodiment, an example in which the sensor 11 and the feature amount data generation unit 12 are integrated will be described. The sensor 11 and the feature amount data generation unit 12 may be provided as separate devices.

As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include a sensor other than the acceleration sensor 111 and the angular velocity sensor 112. The sensor, other than the acceleration sensor 111 and the angular velocity sensor 112, that can be included in the sensor 11 will not be described.

The acceleration sensor 111 is a sensor that measures acceleration (also referred to as spatial acceleration) in the three axis directions. The acceleration sensor 111 measures acceleration (also referred to as spatial acceleration) as a physical quantity related to the motion of the foot. The acceleration sensor 111 outputs the measured acceleration to the feature amount data generation unit 12. For example, a sensor of a piezoelectric type, a piezoresistive type, a capacitance type, or the like can be used as the acceleration sensor 111. As long as the sensor used as the acceleration sensor 111 can measure acceleration, the measurement method is not limited.

The angular velocity sensor 112 is a sensor that measures angular velocities around the three axes (also referred to as spatial angular velocities). The angular velocity sensor 112 measures an angular velocity (also referred to as a spatial angular velocity) as a physical quantity related to the motion of the foot. The angular velocity sensor 112 outputs the measured angular velocity to the feature amount data generation unit 12. For example, a sensor of a vibration type, a capacitance type, or the like can be used as the angular velocity sensor 112. As long as the sensor used as the angular velocity sensor 112 can measure the angular velocity, the measurement method is not limited.

The sensor 11 is achieved by, for example, an inertial measurement device that measures acceleration and angular velocity. An example of the inertial measurement device is an inertial measurement unit (IMU). The IMU includes the acceleration sensor 111 that measures acceleration in three axis directions and the angular velocity sensor 112 that measures angular velocities around the three axes. The sensor 11 may be achieved by an inertial measurement device such as a vertical gyro (VG) or an attitude heading (AHRS). The sensor 11 may be achieved by a global positioning system/inertial navigation system (GPS/INS). The sensor 11 may be achieved by a device other than the inertial measurement device as long as it can measure a physical quantity related to the motion of the foot.

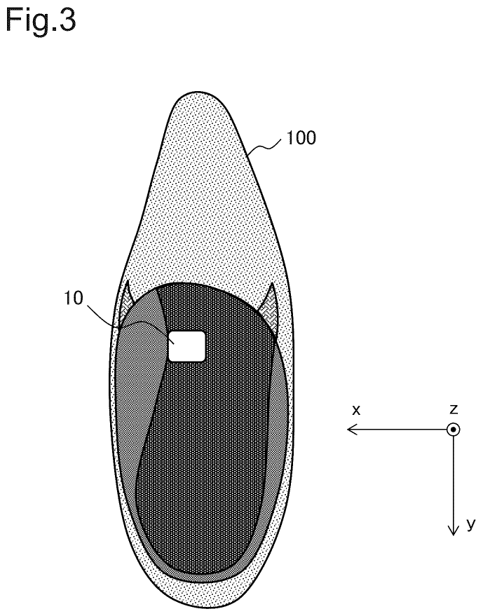

FIG. 3 is a conceptual diagram illustrating an example in which the gait measurement device 10 is disposed in a shoe 100 of the right foot. In the example of FIG. 3, the gait measurement device 10 is installed at a position related to the back side of the arch of foot. For example, the gait measurement device 10 is disposed at an insole inserted into the shoe 100. For example, the gait measurement device 10 may be disposed on the bottom face of the shoe 100. For example, the gait measurement device 10 may be embedded in the main body of the shoe 100. The gait measurement device 10 may be detachable from the shoe 100 or may not be detachable from the shoe 100. The gait measurement device 10 may be installed at a position other than the back side of the arch of the foot as long as the sensor data related to the motion of the foot can be measured. The gait measurement device 10 may be installed at a sock worn by the user or a decorative article such as an anklet worn by the user. The gait measurement device 10 may be directly attached to the foot or may be embedded in the foot. FIG. 3 illustrates an example in which the gait measurement device 10 is installed at the shoe 100 of the right foot. The gait measurement device 10 may be installed at the shoes 100 of both feet.

In the example of FIG. 3, a local coordinate system including an x axis in the horizontal direction, a y axis in the front-rear direction, and a z axis in the vertical direction is set with the gait measurement device 10 (sensor 11) as a reference. In the x axis, the left side is positive, in the y axis, the rear side is positive, and in the z axis, the upper side is positive. The direction of the axis set in the sensor 11 may be the same for the left and right feet, or may be different for the left and right feet. For example, in a case where the sensors 11 produced with the same specifications are disposed in the left and right shoes 100, the vertical directions (directions in the Z axis direction) of the sensors 11 disposed in the left and right shoes 100 are the same. In this case, the three axes of the local coordinate system set in the sensor data derived from the left foot and the three axes of the local coordinate system set in the sensor data derived from the right foot are the same for the left and right feet.

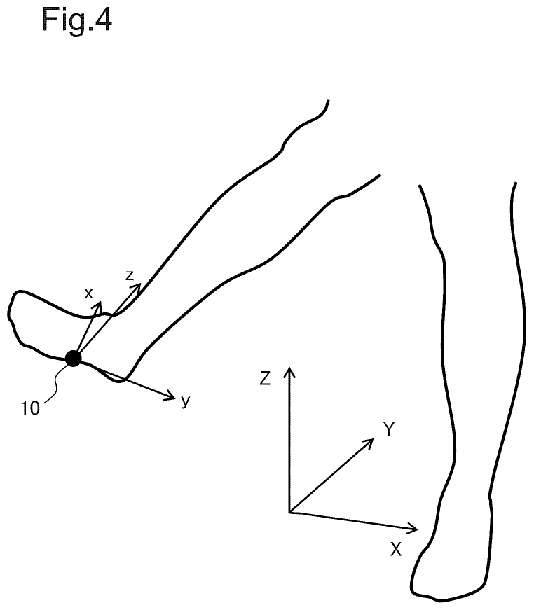

FIG. 4 is a conceptual diagram for describing a local coordinate system (x axis, y axis, z axis) set in the gait measurement device 10 (sensor 11) installed at the back side of the arch of foot and a world coordinate system (X axis, Y axis, Z axis) set with respect to the ground. In the world coordinate system (X axis, Y axis, Z axis), in a state where the user facing the traveling direction is standing upright, a lateral direction of the user is set to an X axis direction (leftward direction is positive), a back face direction of the user is set to a Y axis direction (rearward direction is positive), and a gravity direction is set to a Z axis direction (vertically upward direction is positive). The example of FIG. 4 conceptually illustrates the relationship between the local coordinate system (x axis, y axis, z axis) and the world coordinate system (X axis, Y axis, Z axis), and does not accurately illustrate the relationship between the local coordinate system and the world coordinate system that varies depending on a gait of the user.

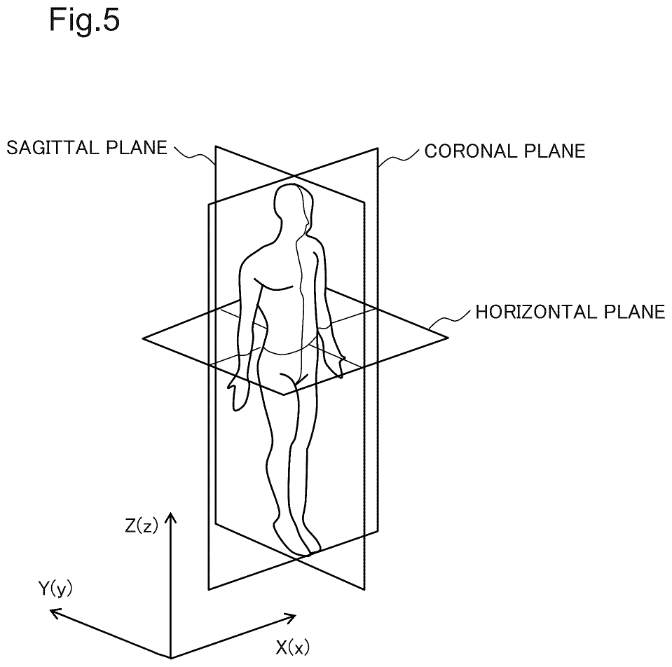

FIG. 5 is a conceptual diagram for describing a face (also referred to as a human body surface) set for the human body. In the present example embodiment, a sagittal plane dividing the body into left and right, a coronal plane dividing the body into front and rear, and a horizontal plane dividing the body horizontally are defined. As illustrated in FIG. 5, the world coordinate system and the local coordinate system coincide with each other in a state in which the user is standing upright with the center line of the foot being directed in the traveling direction. In the present example embodiment, rotation in the sagittal plane with the x axis as a rotation axis is defined as roll, rotation in the coronal plane with the y axis as a rotation axis is defined as pitch, and rotation in the horizontal plane with the z axis as a rotation axis is defined as yaw. A rotation angle in a sagittal plane with the x axis as a rotation axis is defined as a roll angle, a rotation angle in a coronal plane with the y axis as a rotation axis is defined as a pitch angle, and a rotation angle in a horizontal plane with the z axis as a rotation axis is defined as a yaw angle.

As illustrated in FIG. 2, the feature amount data generation unit 12 (also referred to as a feature amount data generation device) includes an acquisition unit 121, a normalization unit 122, an extraction unit 123, a generation unit 125, and a feature amount data output unit 127. For example, the feature amount data generation unit 12 is achieved by a microcomputer or a microcontroller that performs overall control and data processing of the gait measurement device 10. For example, the feature amount data generation unit 12 includes a central processing unit (CPU), a random access memory (RAM), a read only memory (ROM), a flash memory, and the like. The feature amount data generation unit 12 controls the acceleration sensor 111 and the angular velocity sensor 112 to measure the angular velocity and the acceleration. For example, the feature amount data generation unit 12 may be mounted in a mobile terminal (not illustrated) carried by a subject (user).

The acquisition unit 121 acquires acceleration in three axis directions from the acceleration sensor 111. The acquisition unit 121 acquires angular velocities around three axes from the angular velocity sensor 112. For example, the acquisition unit 121 performs analog-to-digital conversion (AD conversion) on the acquired physical quantities (analog data) such as angular velocity and acceleration. The physical quantity (analog data) measured by each of the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to store the sensor data in a storage unit (not illustrated). The sensor data includes at least acceleration data converted into digital data and angular velocity data converted into digital data. The acceleration data includes acceleration vectors in the three axis directions. The angular velocity data includes angular velocity vectors around three axes. The acceleration data and the angular velocity data are associated with acquisition time of the data. The acquisition unit 121 may add correction such as a mounting error, temperature correction, and linearity correction to the acceleration data and the angular velocity data.

The normalization unit 122 acquires sensor data from the acquisition unit 121. The normalization unit 122 extracts time series data (also referred to as gait waveform data) for one gait cycle from the time series data of the acceleration in the three axis direction and the angular velocities around the three axes included in the sensor data. The normalization unit 122 normalizes (also referred to as first normalization) the time of the extracted gait waveform data for one gait cycle to a gait cycle of 0 to 100% (percent). Timing such as 1% or 10% included in the 0 to 100% gait cycle is also referred to as a gait phase. The normalization unit 122 normalizes (also referred to as second normalization) the first normalized gait waveform data for one gait cycle in such a way that the stance phase is 60% and the swing phase is 40%. The stance phase is a period in which at least part of the back side of the foot is in contact with the ground. The swing phase is a period in which the back side of the foot is away from the ground. When the gait waveform data is second normalized, it is possible to suppress the deviation of the gait phase from which the feature amount is extracted from fluctuating due to the influence of disturbance.

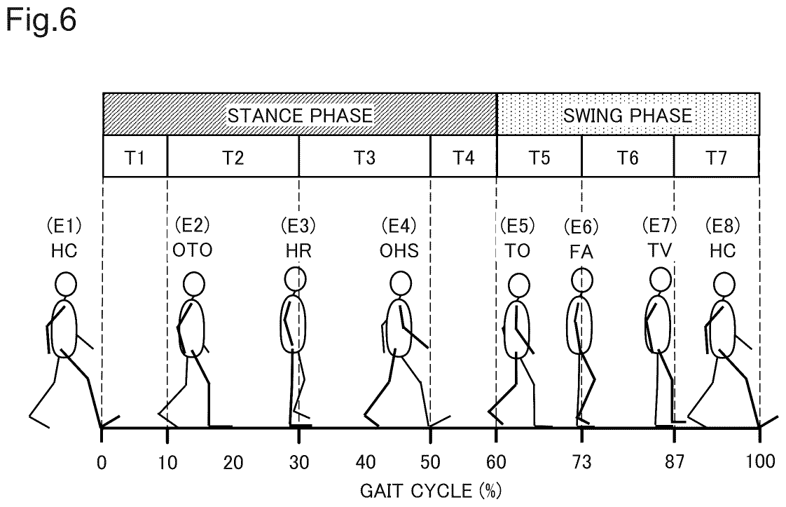

FIG. 6 is a conceptual diagram for describing one gait cycle with the right foot as a reference. One gait cycle based on the left foot is also similar to that of the right foot. The horizontal axis of FIG. 6 is one gait cycle of the right foot with a time point at which the heel of the right foot lands on the ground as a starting point and a time point at which the heel of the right foot next lands on the ground as an ending point. The horizontal axis in FIG. 6 is first normalized with one gait cycle as 100%. The horizontal axis of FIG. 6 is second normalized in such a way that the stance phase is 60% and the swing phase is 40%. The one gait cycle of one foot is roughly divided into a stance phase in which at least part of the back side of the foot is in contact with the ground and the swing phase in which the back side of the foot is away from the ground. The stance phase is further subdivided into a load response period T1, a mid-stance period T2, a terminal stance period T3, and a pre-swing period T4. The swing phase is further subdivided into an initial swing period T5, a mid-swing period T6, and a terminal swing period T7. FIG. 6 is an example, and does not limit the periods constituting one gait cycle, the names of these periods, and the like.

As illustrated in FIG. 6, in gait, a plurality of events (also referred to as gait events) occur. E1 represents an event (heel contact (HC)) in which the heel of the right foot touches the ground. E2 represents an event (opposite toe off (OTO)) in which the toe of the right foot is away from the ground while the sole of the left foot is grounded. E3 represents an event (heel rise (HR)) in which the heel of the right foot is lifted while the sole of the right foot is grounded. E4 represents an event (opposite heel strike (OHS)) in which the heel of the left foot is grounded. E5 represents an event (toe off (TO)) in which the toe of the left foot is away from the ground while the sole of the right foot is grounded. E6 represents an event (foot adjacent (FA)) in which the left foot and the right foot cross each other while the sole of the left foot is grounded. E7 represents an event (tibia vertical (TV)) in which the tibia of the left foot is substantially perpendicular to the ground in a state where the sole of the right foot is in contact with the ground. E8 represents an event (heel contact (HC)) in which the heel of the right foot is grounded. E8 corresponds to the ending point of the gait cycle starting from E1 and corresponds to the starting point of the next gait cycle. FIG. 6 is an example, and does not limit events that occur during gait or names of these events.

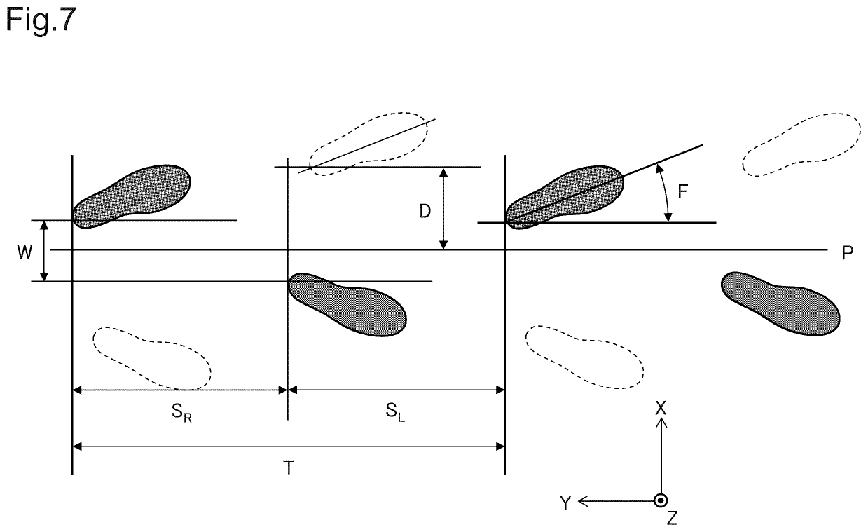

FIG. 7 is a conceptual diagram for describing an example of a gait parameter. FIG. 7 illustrates a right foot step length SR, a left foot step length SL, a stride length T, a stride width W, a foot angle F, and a circumduction amount D. FIG. 7 illustrates a traveling axis P that is parallel to the axis (Y axis) in the traveling direction and corresponds to a trajectory connecting the middle of the left and right legs. The right foot step length SR is a difference in the Y-coordinate between the heel of the right foot and the heel of the left foot when the state in which the sole of the left foot is in contact with the ground transitions to the state in which the heel of the right foot swung out in the traveling direction lands on the ground. The left foot step length SL is a difference in the Y-coordinate between the heel of the left foot and the heel of the right foot when the state in which the sole of the right foot is in contact with the ground transitions to the state in which the heel of the left foot swung out in the traveling direction lands on the ground. The stride length T is the sum of the right foot step length SR and the left foot step length SL. The stride width W is a distance between the right foot and the left foot. In FIG. 7, the stride width W is a difference between the X coordinate of the center line of the heel of the right foot in a grounded state and the X coordinate of the center line of the heel of the left foot in a grounded state. The foot angle F is an angle formed by the center line of the foot and the traveling direction (Y axis) in a state where the sole of the foot is in contact with the ground. In the present example embodiment, the foot angle in a state where the foot is in contact with the ground is evaluated in the stance phase. The circumduction amount D is a distance between the traveling axis P and the foot at a timing when the center axis of the foot is farthest from the traveling axis P in the swing phase. In the present example embodiment, since the length of the lower limb affects the circumduction amount D, the circumduction amount D is normalized by the height.

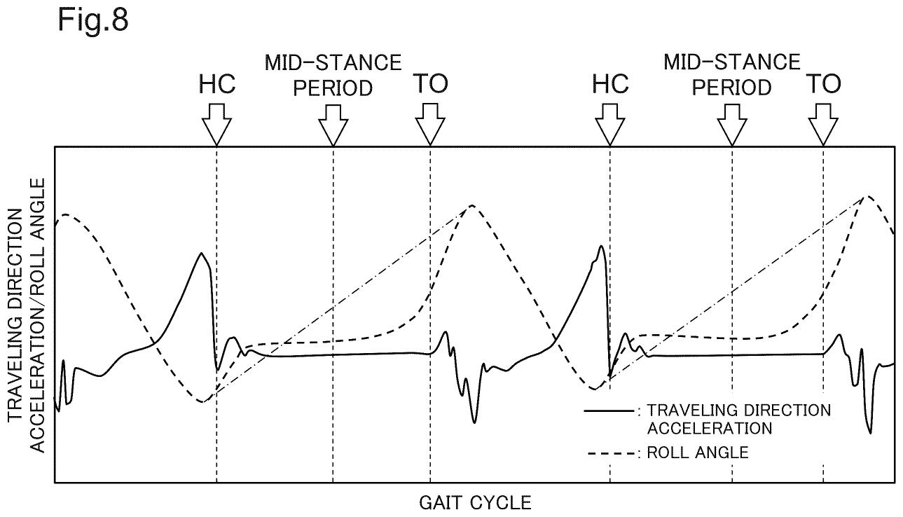

FIG. 8 is a diagram for describing an example of detecting the heel contact HC and the toe off TO from the time series data (solid line) of the acceleration in the traveling direction (Y direction acceleration). The timing of the heel contact HC is the timing of the minimum peak immediately after the maximum peak appearing in the time series data of the acceleration in the traveling direction (Y direction acceleration). The maximum peak serving as a mark of the timing of the heel contact HC corresponds to the maximum peak of the gait waveform data for one gait cycle. A section between the consecutive heel contacts HC is one gait cycle. The timing of the toe off TO is the rising timing of the maximum peak appearing after the period of the stance phase in which the fluctuation does not appear in the time series data of the acceleration in the traveling direction (Y direction acceleration). FIG. 8 also illustrates time series data (broken line) of the roll angle (angular velocity around the X axis). The timing at the midpoint between the timing at which the roll angle is minimum and the timing at which the roll angle is maximum corresponds to the mid-stance period. For example, parameters (also referred to as gait parameters) such as the gait speed, the stride length, the circumduction, the incycloduction/excycloduction, and the plantarflexion/dorsiflexion can also be obtained with the mid-stance period as a reference.

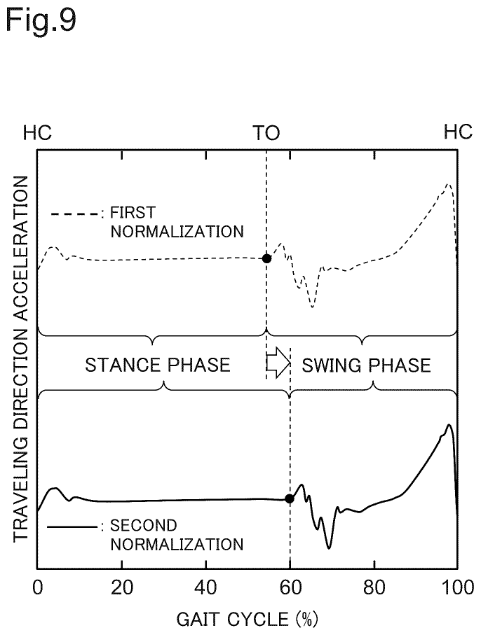

FIG. 9 is a diagram for describing an example of the gait waveform data normalized by the normalization unit 122. The normalization unit 122 detects the heel contact HC and the toe off TO from the time series data of the acceleration in the traveling direction (Y direction acceleration). The normalization unit 122 extracts a section between consecutive heel contacts HC as gait waveform data for one gait cycle. The normalization unit 122 converts the horizontal axis (time axis) of the gait waveform data for one gait cycle into a gait cycle of 0 to 100% by the first normalization. In FIG. 9, the gait waveform data after the first normalization is indicated by a broken line. In the gait waveform data (broken line) after the first normalization, the timing of the toe off TO is shifted from 60%.

In the example of FIG. 9, the normalization unit 122 normalizes a section from the heel contact HC in which the gait phase is 0% to the toe off TO subsequent to the heel contact HC to 0 to 60%. The normalization unit 122 normalizes a section from the toe off TO to the heel contact HC in which the gait phase subsequent to the toe off TO is 100% to 60 to 100%. As a result, the gait waveform data for one gait cycle is normalized to a section (stance phase) in which the gait cycle is 0 to 60% and a section (swing phase) in which the gait cycle is 60 to 100%. In FIG. 9, the gait waveform data after the second normalization is indicated by a solid line. In the gait waveform data (solid line) after the second normalization, the timing of the toe off TO coincides with 60%.

FIGS. 8 to 9 illustrate examples in which the gait waveform data for one gait cycle is extracted/normalized based on the acceleration in the traveling direction (Y direction acceleration). With respect to acceleration/angular velocity other than the acceleration in the traveling direction (Y direction acceleration), the normalization unit 122 extracts/normalizes gait waveform data for one gait cycle in accordance with the gait cycle of the acceleration in the traveling direction (Y direction acceleration). The normalization unit 122 may generate time series data of angles around three axes by integrating time series data of angular velocities around the three axes. In this case, the normalization unit 122 extracts/normalizes the gait waveform data for one gait cycle in accordance with the gait cycle of the acceleration in the traveling direction (Y direction acceleration) with respect to the angles around the three axes.

The normalization unit 122 may extract/normalize the gait waveform data for one gait cycle based on acceleration/angular velocity other than the acceleration in the traveling direction (Y direction acceleration) (not illustrated). For example, the normalization unit 122 may detect the heel contact HC and the toe off TO from the time series data of the vertical acceleration (Z direction acceleration). The timing of the heel contact HC is a timing of a steep minimum peak appearing in the time series data of the vertical acceleration (Z direction acceleration). At the timing of the steep minimum peak, the value of the vertical acceleration (Z direction acceleration) is substantially zero. The minimum peak serving as a mark of the timing of the heel contact HC corresponds to the minimum peak of the gait waveform data for one gait cycle. A section between the consecutive heel contacts HC is one gait cycle. The timing of the toe off TO is a timing of an inflection point in the middle of gradually increasing after the time series data of the vertical acceleration (Z direction acceleration) passes through a section with a small fluctuation after the maximum peak immediately after the heel contact HC. The normalization unit 122 may extract/normalize the gait waveform data for one gait cycle based on both the acceleration in the traveling direction (Y direction acceleration) and the vertical acceleration (Z direction acceleration). The normalization unit 122 may extract/normalize the gait waveform data for one gait cycle based on acceleration, angular velocity, angle, and the like other than the acceleration in the traveling direction (Y direction acceleration) and the vertical acceleration (Z direction acceleration).

The extraction unit 123 acquires gait waveform data for one gait cycle normalized by the normalization unit 122. The extraction unit 123 extracts a feature amount used for estimating the static balance from the gait waveform data for one gait cycle. The extraction unit 123 extracts a feature amount for each gait phase cluster from a gait phase cluster obtained by integrating temporally continuous gait phases based on a preset condition. The gait phase cluster includes at least one gait phase. The gait phase cluster also includes a single gait phase. The gait waveform data and the gait phase from which the feature amount used for estimating the static balance is extracted will be described later.

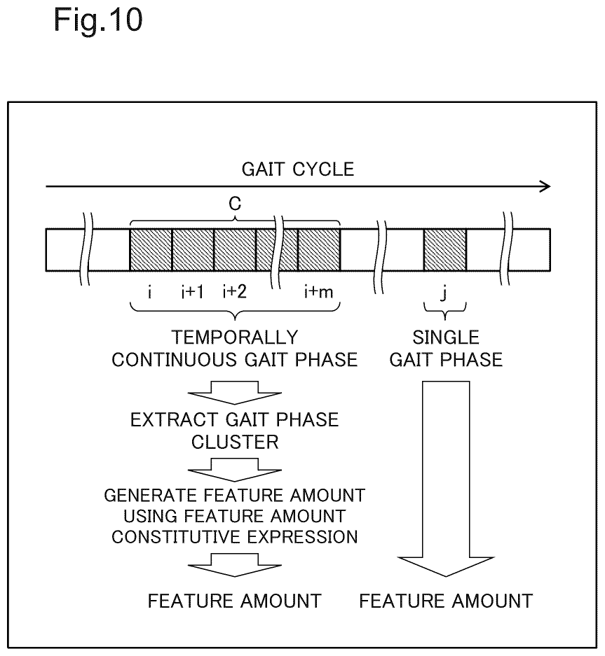

FIG. 10 is a conceptual diagram for describing extraction of a feature amount for estimating static balance from gait waveform data for one gait cycle. For example, the extraction unit 123 extracts temporally continuous gait phases i to i+m as a gait phase cluster C (i and m are natural numbers). The gait phase cluster C includes m gait phases (components). That is, the number of gait phases (components) (also referred to as the number of components) constituting the gait phase cluster C is m. FIG. 10 illustrates an example in which the gait phase has an integer value, but the gait phase may be subdivided into decimal places. When the gait phase is subdivided into decimal places, the number of components of the gait phase cluster C is a number related to the number of data points in the section of the gait phase cluster. The extraction unit 123 extracts a feature amount from each of the gait phases i to i+m. In a case where the gait phase cluster C includes a single gait phase j, the extraction unit 123 extracts a feature amount from the single gait phase j (j is a natural number).

The generation unit 125 applies the feature amount constitutive expression to the feature amount (first feature amount) extracted from each of the gait phases constituting the gait phase cluster to generate the feature amount (second feature amount) of the gait phase cluster. The feature amount constitutive expression is a preset calculation expression for generating the feature amount of the gait phase cluster. For example, the feature amount constitutive expression is a calculation expression related to four arithmetic operations. For example, the second feature amount calculated using the feature amount constitutive expression is an integral average value, an arithmetic average value, an inclination, a variation, or the like of the first feature amount in each gait phase included in the gait phase cluster. For example, the generation unit 125 applies a calculation expression for calculating the inclination and the variation of the first feature amount extracted from each of the gait phases constituting the gait phase cluster as the feature amount constitutive expression. For example, in a case where the gait phase cluster is configured by a single gait phase, it is not possible to calculate the inclination and the variation, and thus, it is sufficient to use a feature amount constitutive expression for calculating an integral average value, an arithmetic average value, or the like.

The feature amount data output unit 127 outputs the feature amount data for each gait phase cluster generated by the generation unit 125. The feature amount data output unit 127 outputs the generated feature amount data of the gait phase cluster to the static balance estimation device 13 that uses the feature amount data.

### [Static Balance Estimation Device]

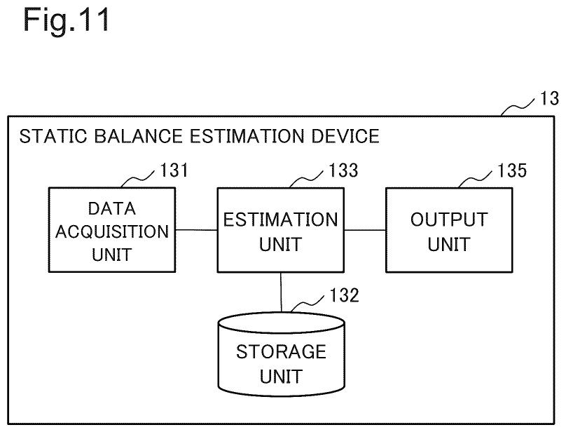

FIG. 11 is a block diagram illustrating an example of a configuration of the static balance estimation device 13. The static balance estimation device 13 includes a data acquisition unit 131, a storage unit 132, an estimation unit 133, and an output unit 135.

The data acquisition unit 131 acquires feature amount data from the gait measurement device 10. The data acquisition unit 131 outputs the received feature amount data to the estimation unit 133. The data acquisition unit 131 may receive the feature amount data from the gait measurement device 10 via a wire such as a cable, or may receive the feature amount data from the gait measurement device 10 via wireless communication. For example, the data acquisition unit 131 is configured to receive the feature amount data from the gait measurement device 10 via a wireless communication function (not illustrated) conforming to a standard such as Bluetooth (registered trademark) or Wi-Fi (registered trademark). The communication function of the data acquisition unit 131 may conform to a standard other than Bluetooth (registered trademark) or Wi-Fi (registered trademark).

The storage unit 132 stores an estimation model for estimating the one leg standing position time as the static balance using the feature amount data extracted from the gait waveform data. The storage unit 132 stores an estimation model that has machine learned the relationship between the feature amount data regarding the one leg standing position time of the plurality of subjects and the one leg standing position time. For example, the storage unit 132 stores an estimation model for estimating the one leg standing position time, the estimation model on which machine learning have been performed for a plurality of subjects. The one leg standing position time is affected by age and height. Therefore, the storage unit 132 may store an estimation model according to attribute data regarding at least one of age and height.

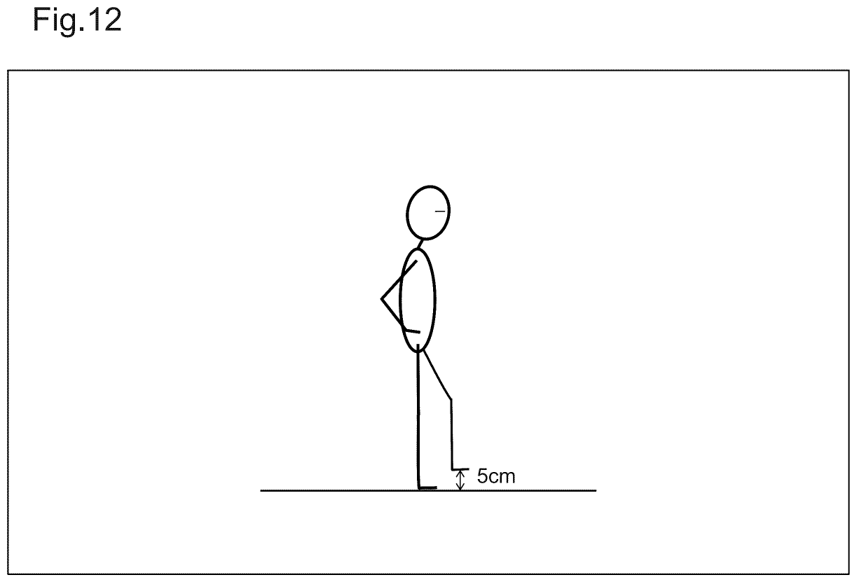

FIG. 12 is a conceptual diagram for describing a one leg standing position test. FIG. 12 illustrates a state in which the subject closes the eyes and raises one leg by 5 cm (centimeters) from the ground. In the present example embodiment, the closed eye one leg standing position test is taken as an example. The method of the present example embodiment can also be applied to a one foot standing position test other than the closed eye one leg standing position test, such as the open eye one leg standing position test performed with the eyes open.

The static balance can be evaluated according to the time (also referred to as a closed eye one leg standing position time) during which the closed eye one leg standing position can be maintained. When the closed eye one leg standing position time is equal to or more than 30 seconds, the static balance is high and the possibility of falling is low. When the closed eye one leg standing position time is in the range of 15 to 30 seconds, the static balance is low, and there is a possibility of falling. When the closed eye one leg standing position time is less than 15 seconds, the static balance is rather low and the possibility of falling is very high. The evaluation criterion of the static balance according to the closed eye one leg standing position time described herein is a guide, and may be set according to the situation. For example, the evaluation criterion of the static balance according to the closed eye one leg standing position time varies depending on the previous disease of the subject. In the case of the one leg standing position test other than the closed eye one leg standing position test, the evaluation criterion may be set according to these tests. Hereinafter, the time during which the one leg standing position can be maintained, including the closed eye one leg standing position time, is referred to as a one leg standing position time.

The estimation model may be stored in the storage unit 132 at the time of factory shipment of a product, calibration before the user uses the static balance estimation system 1, or the like. For example, an estimation model stored in a storage device such as an external server may be used. In this case, the estimation model may be configured to be used via an interface (not illustrated) connected to the storage device.

The estimation unit 133 acquires the feature amount data from the data acquisition unit 131. The estimation unit 133 estimates the one leg standing position time as the static balance using the acquired feature amount data. The estimation unit 133 inputs the feature amount data to the estimation model stored in the storage unit 132. The estimation unit 133 outputs an estimation result related to the static balance (one leg standing position time) output from the estimation model. In a case where an estimation model stored in an external storage device constructed in a cloud, a server, or the like is used, the estimation unit 133 is configured to use the estimation model via an interface (not illustrated) connected to the storage device.

The output unit 135 outputs the estimation result of the static balance by the estimation unit 133. For example, the output unit 135 displays the estimation result of the static balance on the screen of the mobile terminal of the subject (user). For example, the output unit 135 outputs the estimation result to an external system or the like that uses the estimation result. The use of the static balance output from the static balance estimation device 13 is not particularly limited.

For example, the static balance estimation device 13 is connected to an external system or the like constructed in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile telephone. For example, the static balance estimation device 13 is connected to a mobile terminal via a wire such as a cable. For example, the static balance estimation device 13 is connected to a mobile terminal via wireless communication. For example, the static balance estimation device 13 is connected to a mobile terminal via a wireless communication function (not illustrated) conforming to a standard such as Bluetooth (registered trademark) or Wi-Fi (registered trademark). The communication function of the static balance estimation device 13 may conform to a standard other than Bluetooth (registered trademark) or Wi-Fi (registered trademark). The static balance estimation result may be used by an application installed at the mobile terminal. In this case, the mobile terminal executes processing using the estimation result by application software or the like installed in the mobile terminal.

### [Estimation of One Leg Standing Position Time]

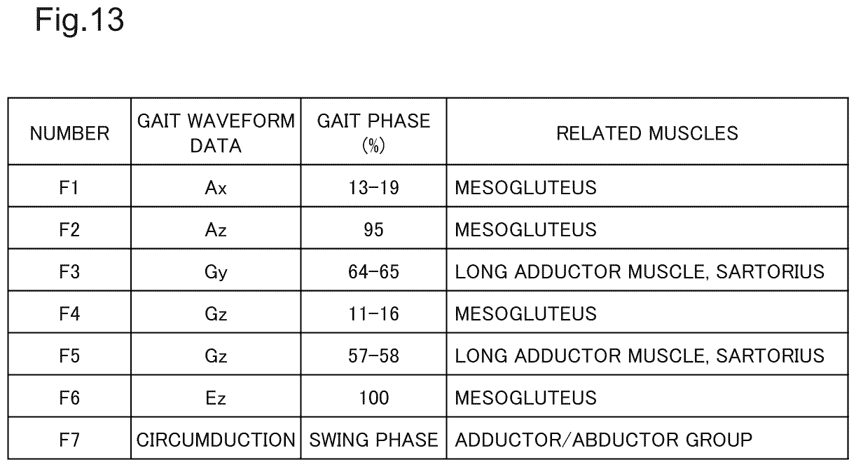

Next, the correlation between the one leg standing position time and the feature amount data will be described with reference to a verification example. FIG. 13 is a correspondence table summarizing feature amounts used for estimating the one leg standing position time. The correspondence table of FIG. 13 associates the number of the feature amount, the gait waveform data from which the feature amount is extracted, the gait phase (%) from which the gait phase cluster is extracted, and the related muscle with each other. The one leg standing position time is correlated with the mesogluteus, the long adductor muscle, the sartorius, and the adductor/abductor group. Therefore, the feature amounts F1 to F7 extracted from the gait phase in which these features appear are used to estimate the one leg standing position time.

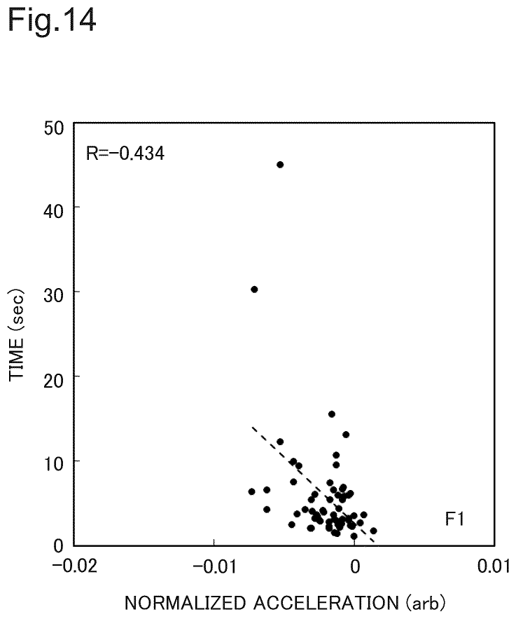

FIGS. 14 to 20 are verification results of the correlation between the one leg standing position time and the feature amount data. FIGS. 14 to 20 illustrate the results of verification performed on a total of 62 subjects including 27 males and 35 females aged 60 to 85 years. FIGS. 14 to 20 illustrate a result of verifying the correlation between the estimation value estimated using the feature amount extracted according to the gait with the footwear on which the gait measurement device 10 is mounted and the measurement value (true value) of the one leg standing position time.

The feature amount F1 is extracted from a section of the gait phase 13 to 19% of the gait waveform data Ax regarding the time series data of the lateral acceleration (X direction acceleration). The gait phase 13 to 19% is included in the mid-stance period T2. The feature amount F1 mainly includes a feature related to the motion of the mesogluteus. FIG. 14 is a verification result of the correlation between the feature amount F1 and the one leg standing position time. The horizontal axis of the graph of FIG. 14 is the normalized acceleration. The correlation coefficient R between the feature amount F1 and the one leg standing position time was −0.434.

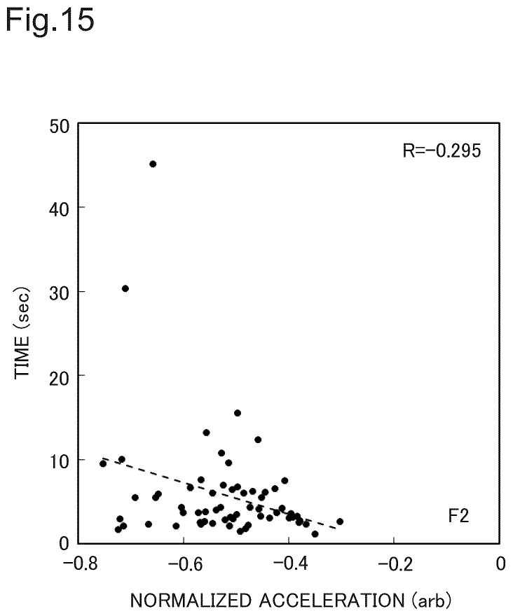

The feature amount F2 is extracted from a section of the gait phase 95% of the gait waveform data Az regarding the time series data of the vertical acceleration (Z direction acceleration). The gait phase 95% is the end of the terminal swing period T7. The feature amount F2 mainly includes a feature related to the motion of the mesogluteus. FIG. 15 is a verification result of the correlation between the feature amount F2 and the one leg standing position time. The horizontal axis of the graph of FIG. 15 is the normalized acceleration. The correlation coefficient R between the feature amount F2 and the one leg standing position time was −0.295.

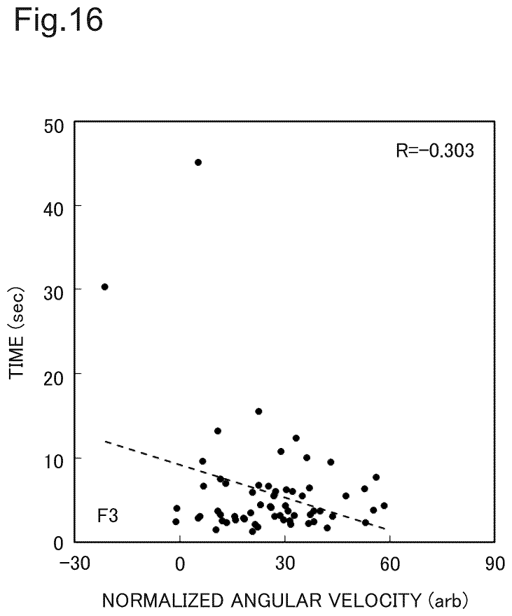

The feature amount F3 is extracted from the section of the gait phase 64 to 65% of the gait waveform data Gy regarding the time series data of the angular velocity in the coronal plane (around the Y axis). The gait phase 64 to 65% is included in the initial swing period T5. The feature amount F3 mainly includes features related to the motion of the long adductor muscle and the sartorius. FIG. 16 is a verification result of the correlation between the feature amount F3 and the one leg standing position time. The horizontal axis of the graph of FIG. 16 is the normalized angular velocity. The correlation coefficient R between the feature amount F3 and the one leg standing position time was −0.303.

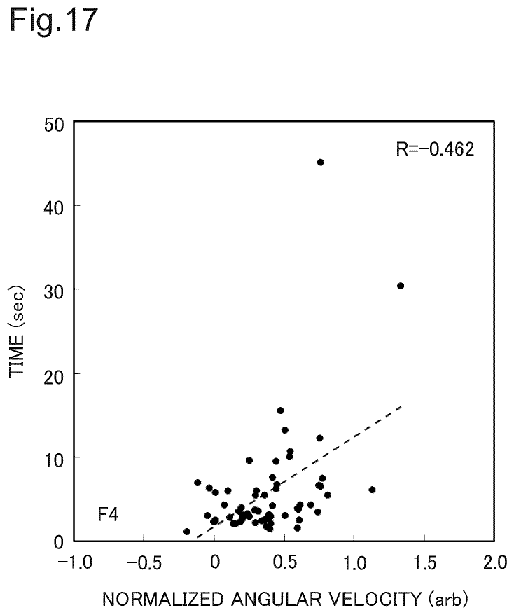

The feature amount F4 is extracted from the section of the gait phase 11 to 16% of the gait waveform data Gz regarding the time series data of the angular velocity in the horizontal plane (around the Z axis). The gait phase 11 to 16% is included in the mid-stance period T2. The feature amount F4 mainly includes a feature related to the motion of the mesogluteus. FIG. 17 is a verification result of the correlation between the feature amount F4 and the one leg standing position time. The horizontal axis of the graph of FIG. 17 is the normalized angular velocity. The correlation coefficient R between the feature amount F4 and the one leg standing position time was −0.462.

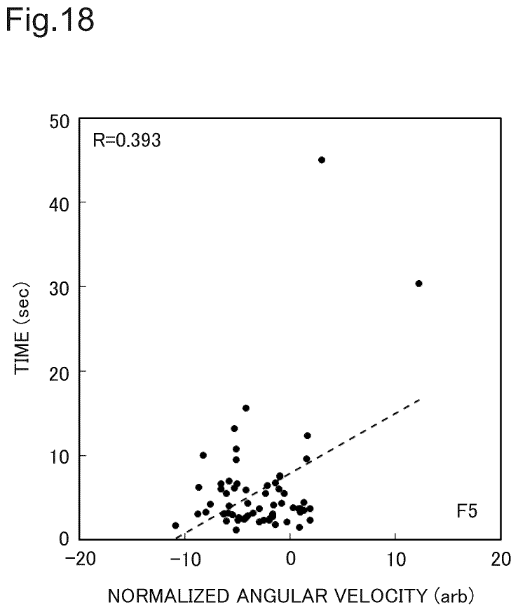

The feature amount F5 is extracted from the section of the gait phase 57 to 58% of the gait waveform data Gz regarding the time series data of the angular velocity in the horizontal plane (around the Z axis). The gait phase 57 to 58% is included in the pre-swing period T4. The feature amount F5 mainly includes a feature related to the motion of the long adductor muscle and the sartorius. FIG. 18 is a verification result of the correlation between the feature amount F5 and the one leg standing position time. The horizontal axis of the graph of FIG. 18 is the normalized angular velocity. The correlation coefficient R between the feature amount F4 and the one leg standing position time was 0.393.

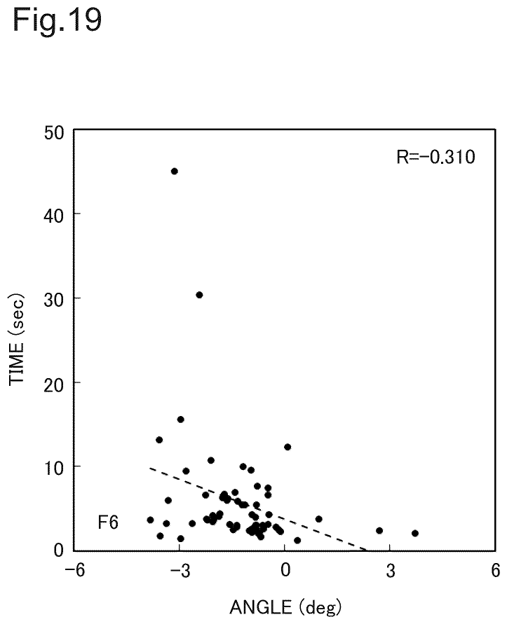

The feature amount F6 is extracted from the section of the gait phase 100% of the gait waveform data Ez regarding the time series data of the angle (posture angle) in the horizontal plane (around the Z axis). The gait phase 100% corresponds to the timing of heel contact when a gait switches from the terminal swing period T7 to the load response period T1. The feature amount of the gait waveform data Ez in the gait phase 100% corresponds to a foot angle in a state where the sole is grounded. The feature amount F6 mainly includes a feature related to the motion of the mesogluteus. FIG. 19 is a verification result of the correlation between the feature amount F6 and the one leg standing position time. The horizontal axis of the graph in FIG. 19 represents an angle (plantar angle) in a horizontal plane. The correlation coefficient R between the feature amount F6 and the one leg standing position time was −0.310. The feature amount F6 is not an essential feature amount for the estimation of the one foot standing position time, but improves the estimation accuracy of the one foot standing position time.

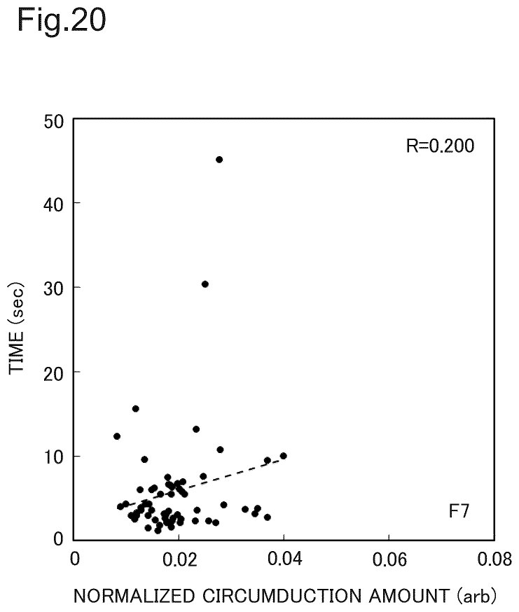

The feature amount F7 is a distance (circumduction amount) between the traveling axis and the foot at a timing when the center axis of the foot is farthest from the traveling axis in the swing phase. The feature amount F7 is a circumduction amount normalized by the height of the subject. The feature amount F7 mainly includes a feature related to the motion of the adductor/abductor group. FIG. 20 is a verification result of the correlation between the feature amount F7 and the one leg standing position time. The horizontal axis of the graph of FIG. 20 is the circumduction amount normalized by the height (normalized circumduction amount). The correlation coefficient R between the feature amount F7 and the one leg standing position time was 0.200.

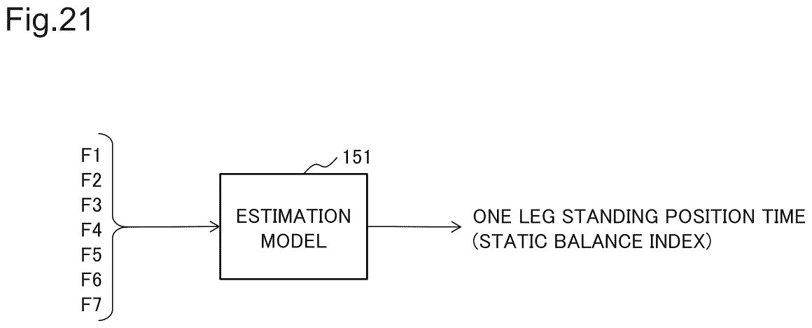

FIG. 21 is a conceptual diagram illustrating an example in which the estimation value of the one leg standing position time is output by inputting the feature amounts F1 to F7 extracted from the sensor data measured along with a gait of the user to an estimation model 151 constructed in advance for estimating the one leg standing position time as the static balance. The estimation model 151 outputs the one leg standing position time that is an index of the static balance according to the inputs of the feature amounts F1 to F7. For example, the estimation model 151 is generated by machine learning using teacher data having the feature amounts F1 to F7 used for estimating the one leg standing position time as explanatory variables and having the one leg standing position time as an objective variable. The estimation result of the estimation model 151 is not limited as long as the estimation result regarding the one leg standing position time that is an index of the static balance is output according to the input of the feature amount data for estimating the one leg standing position time. For example, the estimation model 151 may be a model that estimates the one leg standing position time using attribute data (age, height) as an explanatory variable in addition to the feature amounts F1 to F7 used for estimating the one leg standing position time.

For example, the storage unit 132 stores an estimation model for estimating the one leg standing position time using a multiple regression prediction method. For example, the storage unit 132 stores a parameter for estimating the one leg standing position time using the following Expression 1.

$$one leg standing position time = a 1 \times F 1 + a 2 \times F 2 + a 3 \times F 3 + a 4 \times F 4 + a 5 \times F 5 + a 6 \times F 6 + a 7 \times F 7 + a 0 \qquad ( 1 )$$

In Expression 1 described above, F1, F2, F3, F4, F5, F6, and F7 are feature amounts for respective gait phase clusters used for estimating the one leg standing position time illustrated in the correspondence table in FIG. 13. a1, a2, a3, a4, a5, a6, and a7 are coefficients multiplied by F1, F2, F3, F4, F5, F6, and F7. a0 is a constant term. For example, a0, a1, a2, a3, a4, a5, a6, and a7 are stored in the storage unit 132.

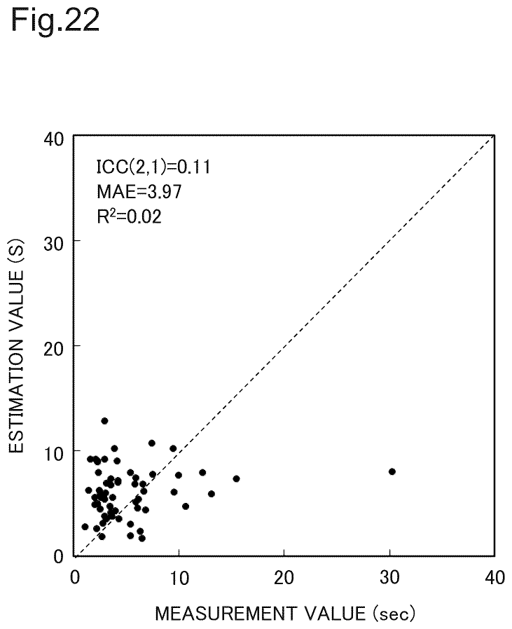

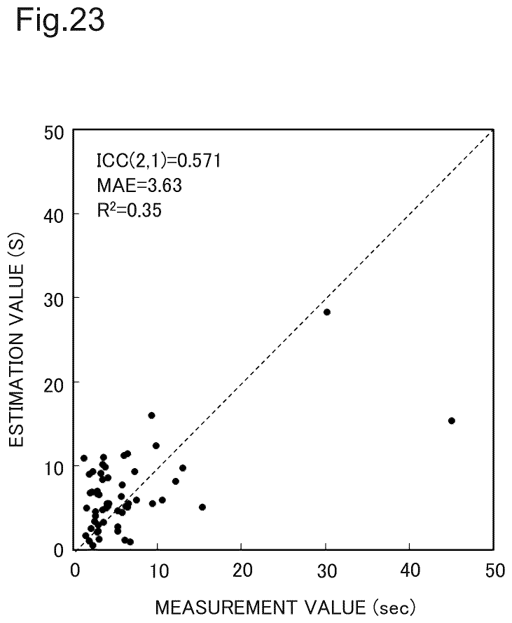

Next, a result of evaluating the estimation model 151 generated using the measurement data of the 62 subjects described above will be described. A verification example (FIG. 22) in which the static balance (one leg standing position time) is estimated using the attribute (including the gait speed) of the subject and a verification example (FIG. 23) in which the static balance (one leg standing position time) is estimated using the feature amount of a gait of the subject are compared. FIGS. 22 and 23 illustrate the results of testing the estimation model generated using the measurement data of 61 people using the measurement data of the remaining 1 person by the Leave-One-Subject-Out (LOSO) method. FIGS. 22 and 23 illustrate the results of performing LOSO on all (62) subjects and associating the prediction value by the test with the measurement value (true value). The test result of LOSO was evaluated by values of an intraclass Correlation Coefficients (ICC), a mean absolute error (MAE), and a determination coefficient R2. As the intraclass correlation coefficient ICC, an intraclass correlation coefficient ICC (2, 1) was used in order to evaluate inter-examiner reliability.

FIG. 22 illustrates a verification result of the estimation model of the comparative example that has machine learned the teacher data with the gender, the age, the height, the weight, and the gait speed as explanatory variables and the one leg standing position time as an objective variable. In the estimation model of the comparative example, the intraclass correlation coefficient ICC (2, 1) was 0.11, the mean absolute error MAE was 3.97, and the determination coefficient R2 was 0.02.

FIG. 23 illustrates a verification result of the estimation model 151 of the present example embodiment that has machine learned teacher data with the feature amounts F1 to F7, the age, and the height as explanatory variables, and the one leg standing position time as an objective variable. In the estimation model 151 of the present example embodiment, the intraclass correlation coefficient ICC (2, 1) was 0.571, the mean absolute error MAE was 3.63, and the determination coefficient R2 was 0.35. That is, the estimation model 151 of the present example embodiment has higher reliability and smaller error than the estimation model of the comparative example, and the objective variable is sufficiently described by the explanatory variable. That is, according to the method of the present example embodiment, it is possible to generate the estimation model 151 that is highly reliable, has a small error, and has the objective variable sufficiently explained by the explanatory variable, as compared with the estimation model using only the attribute and the gait speed.

### (Operation)

Next, an operation of the static balance estimation system 1 will be described with reference to the drawings. The gait measurement device 10 and the static balance estimation device 13 included in the static balance estimation system 1 will be individually described. With respect to the gait measurement device 10, the operation of the feature amount data generation unit 12 included in the gait measurement device 10 will be described.

### [Gait Measurement Device]

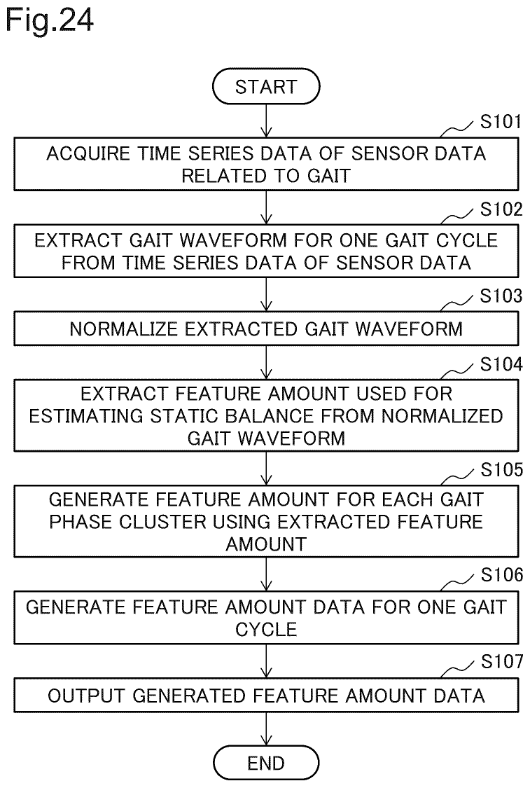

FIG. 24 is a flowchart for describing the operation of the feature amount data generation unit 12 included in the gait measurement device 10. In the description along the flowchart of FIG. 24, the feature amount data generation unit 12 will be described as an operation subject.

In FIG. 24, first, the feature amount data generation unit 12 acquires time series data of sensor data related to the gait (step S101).

Next, the feature amount data generation unit 12 extracts gait waveform data for one gait cycle from the time series data of the sensor data (step S102). The feature amount data generation unit 12 detects the heel contact and the toe off from the time series data of the sensor data. The feature amount data generation unit 12 extracts time series data of a section between consecutive heel contacts as gait waveform data for one gait cycle.

Next, the feature amount data generation unit 12 normalizes the extracted gait waveform data for one gait cycle (step S103). The feature amount data generation unit 12 normalizes the gait waveform data for one gait cycle to a gait cycle of 0 to 100% (first normalization). Furthermore, the feature amount data generation unit 12 normalizes the ratio of the stance phase to the swing phase in the gait waveform data subjected to the first normalization for one gait cycle to 60:40 (second normalization).

Next, the feature amount data generation unit 12 extracts a feature amount from the gait phase used for estimating the static balance with respect to the normalized gait waveform (step S104). For example, the feature amount data generation unit 12 extracts a feature amount input to an estimation model constructed in advance.

Next, the feature amount data generation unit 12 generates a feature amount for each gait phase cluster using the extracted feature amount (step S105).

Next, the feature amount data generation unit 12 integrates the feature amounts for respective gait phase clusters to generate feature amount data for one gait cycle (step S106).

Next, the feature amount data generation unit 12 outputs the generated feature amount data to the static balance estimation device 13 (step S107).

### [Static Balance Estimation Device]

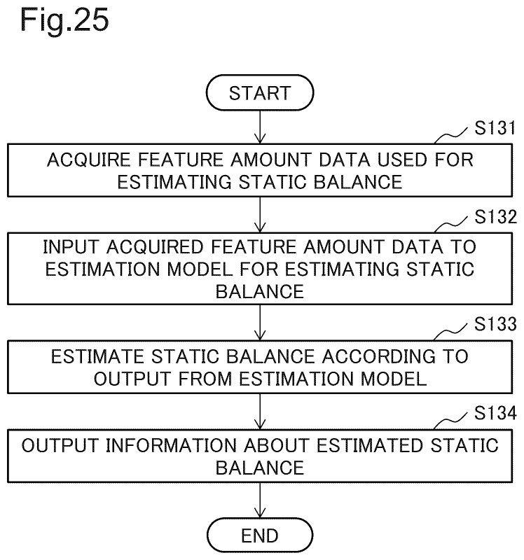

FIG. 25 is a flowchart for describing the operation of the static balance estimation device 13. In the description along the flowchart of FIG. 25, the static balance estimation device 13 will be described as an operation subject.

In FIG. 25, first, the static balance estimation device 13 acquires the feature amount data generated using the sensor data regarding the gait (step S131).

Next, the static balance estimation device 13 inputs the acquired feature amount data to an estimation model for estimating the static balance (one leg standing position time) (step S132).

Next, the static balance estimation device 13 estimates the static balance of the user according to the output (estimation value) from the estimation model (step S133). For example, the static balance estimation device 13 estimates the one leg standing position time of the user as the static balance.

Next, the static balance estimation device 13 outputs information related to the estimated static balance (step S134). For example, the static balance is output to a terminal device (not illustrated) carried by the user. For example, the static balance is output to a system that performs a process using the static balance.

### Application Example

Next, an application example according to the present example embodiment will be described with reference to the drawings. In the following application example, an example in which the function of the static balance estimation device 13 installed in the mobile terminal carried by the user estimates the information related to the static balance using the feature amount data measured by the gait measurement device 10 disposed at the shoe will be described.

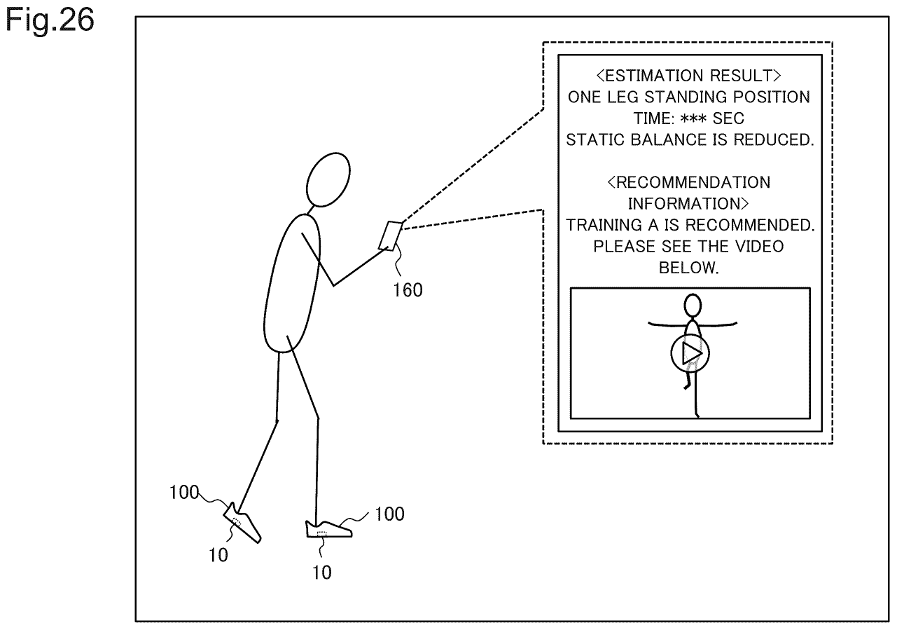

FIG. 26 is a conceptual diagram illustrating an example in which the estimation result by the static balance estimation device 13 is displayed on the screen of a mobile terminal 160 carried by the user walking while wearing the shoes 100 at which the gait measurement devices 10 are disposed. FIG. 26 is an example in which information according to the estimation result of the static balance using the feature amount data according to the sensor data measured while the user is walking is displayed on the screen of the mobile terminal 160.

FIG. 26 illustrates an example in which information related to the estimation value of the one leg standing position time that is the static balance is displayed on the screen of the mobile terminal 160. In the example of FIG. 26, the estimation value of the one leg standing position time is displayed on the display unit of the mobile terminal 160 as the estimation result of the static balance. In the example of FIG. 26, information related to the estimation result of the static balance of “Static balance is reduced.” is displayed on the display unit of the mobile terminal 160 according to the estimation value of the one leg standing position time that is the static balance. In the example of FIG. 26, in accordance with the estimation value of the one leg standing position time that is the static balance, recommendation information according to the estimation result of the static balance of “Training A is recommended. Please see the video below.” is displayed on the display unit of the mobile terminal 160. The user who has confirmed the information displayed on the display unit of the mobile terminal 160 can practice training leading to an increase in static balance by exercising with reference to the video of the training A according to the recommendation information.

As described above, the static balance estimation system of the present example embodiment includes the gait measurement device and the static balance estimation device. The gait measurement device includes a sensor and a feature amount data generation unit. The sensor includes an acceleration sensor and an angular velocity sensor. The sensor measures a spatial acceleration with an acceleration sensor. The sensor measures a spatial angular velocity with an angular velocity sensor. The sensor generates sensor data related to the motion of the foot using the measured spatial acceleration and spatial angular velocity. The sensor outputs the generated sensor data to the feature amount data generation unit. The feature amount data generation unit acquires time series data of sensor data related to motion of a foot. The feature amount data generation unit extracts gait waveform data for one gait cycle from the time series data of the sensor data. The feature amount data generation unit normalizes the extracted gait waveform data. The feature amount data generation unit extracts, from a gait phase cluster configured by at least one temporally continuous gait phase, a feature amount used for estimating the static balance from the normalized gait waveform data. The feature amount data generation unit generates feature amount data including the extracted feature amount. The feature amount data generation unit outputs the generated feature amount data.

The static balance estimation device includes a data acquisition unit, a storage unit, an estimation unit, and an output unit. The data acquisition unit acquires feature amount data including a feature amount used for estimating the static balance of the user, the feature amount being extracted from the feature of a gait of the user. The storage unit stores the estimation model that outputs the static balance index according to the input of the feature amount data. The estimation unit inputs the acquired feature amount data to the estimation model. The estimation unit estimates the static balance of the user according to the static balance index output from the estimation model. The output unit outputs information related to the estimated static balance.

The static balance estimation system of the present example embodiment estimates the static balance of the user using the feature amount extracted from the feature of a gait of the user. Therefore, according to the static balance estimation system of the present example embodiment, the static balance can be appropriately estimated in daily life without using an instrument for measuring the static balance.

In an aspect of the present example embodiment, the data acquisition unit acquires the feature amount data including the feature amount extracted from the gait waveform data generated using the time series data of the sensor data regarding the motion of the foot. The data acquisition unit acquires feature amount data including a feature amount used to estimate a performance value of the one leg standing position test (one leg standing position time) as the static balance index. According to the present aspect, by using the sensor data regarding the motion of the foot, the static balance can be appropriately estimated in daily life without using an instrument for measuring the static balance.

In an aspect of the present example embodiment, the storage unit stores an estimation model generated by machine learning using teacher data related to a plurality of subjects. The estimation model is generated by machine learning using teacher data having a feature amount used for estimating the static balance index as an explanatory variable and the static balance index of each of a plurality of subjects as an objective variable. The estimation unit inputs the feature amount data acquired regarding the user to the estimation model. The estimation unit estimates the static balance of the user according to the static balance index of the user output from the estimation model. According to the present aspect, it is possible to appropriately estimate the static balance in daily life without using an instrument for measuring the static balance.

In an aspect of the present example embodiment, the storage unit stores the estimation model on which machine learning have been performed using the explanatory variables including the attribute data (age, height) of the subject. The estimation unit inputs the feature amount data and the attribute data (age, height) regarding the user to the estimation model. The estimation unit estimates the static balance of the user according to the static balance index of the user output from the estimation model. In the present aspect, the static balance is estimated including attribute data (age, height) that affects the static balance. Therefore, according to the present aspect, the static balance can be measured with higher accuracy.

In an aspect of the present example embodiment, the storage unit stores an estimation model generated by machine learning using teacher data related to a plurality of subjects. The estimation model is a model generated by machine learning using teacher data having a feature amount extracted from the gait waveform data of each of the plurality of subjects as an explanatory variable and a static balance index of each of the plurality of subjects as an objective variable. For example, the explanatory variable includes a feature amount regarding the activity of the mesogluteus extracted from the end of the terminal swing period and the mid-stance period. For example, the explanatory variables include feature amounts related to the activities of the long adductor muscle and sartorius extracted from the pre-swing period and the initial swing period. For example, the explanatory variable includes a feature amount related to the activity of the adductor/abductor group in the swing phase. The estimation unit inputs feature amount data acquired according to a gait of the user to the estimation model. The estimation unit estimates the static balance of the user according to the static balance index of the user output from the estimation model. According to the present aspect, the static balance more suitable for the physical activity can be estimated by using the estimation model that has machine learned the feature amount according to the muscle activity that affects the static balance.

In an aspect of the present example embodiment, the storage unit stores, with respect to a plurality of subjects, an estimation model generated by machine learning using teacher data having a plurality of feature amounts extracted from gait waveform data as explanatory variables and having static balance regarding a static balance index of the subject as an objective variable. For example, the feature amount extracted from the mid-stance period of the gait waveform data of the lateral acceleration is included in the explanatory variable. For example, the feature amount extracted from the end of the terminal swing period of the gait waveform data of the vertical acceleration is included in the explanatory variable. For example, the feature amount extracted from the initial swing period of the gait waveform data of the angular velocity in the coronal plane is included in the explanatory variable. For example, the feature amounts extracted from the mid-stance period and the pre-swing period of the gait waveform data of the angular velocity in the horizontal plane are included in the explanatory variable. For example, the feature amount extracted from the timing of heel contact when a gait switches from the terminal swing period to the load response period of the gait waveform data of the angle in the horizontal plane is included in the explanatory variable. For example, the feature amount related to the circumduction amount in the swing phase is included in the explanatory variable. The data acquisition unit acquires a feature amount in the mid-stance period of the gait waveform data of the lateral acceleration. For example, the data acquisition unit acquires the feature amount at the end of the terminal swing period of the gait waveform data of the vertical acceleration. For example, the data acquisition unit acquires a feature amount of the initial swing period of the gait waveform data of the angular velocity in the coronal plane. For example, the data acquisition unit acquires feature amounts of the mid-stance period and the pre-swing period of the gait waveform data of the angular velocity in the horizontal plane. For example, the data acquisition unit acquires the feature amount at the timing of heel contact when a gait switches from the terminal swing period to the load response period of the gait waveform data of the angle in the horizontal plane. For example, the data acquisition unit acquires a feature amount related to a circumduction amount in the swing phase. The estimation unit inputs the acquired feature amount data to the estimation model. The estimation unit estimates the static balance of the user according to the static balance index of the user output from the estimation model. According to the present aspect, the static balance more suitable for the physical activity can be estimated by using the estimation model that has machine learned the feature amount extracted from the gait waveform data including the feature according to the muscle activity that affects the static balance.

In an aspect of the present example embodiment, the static balance estimation device is mounted in a terminal device having a screen visible by a user. For example, the static balance estimation device displays information related to the static balance estimated according to the motion of the foot of the user on the screen of the terminal device. For example, the static balance estimation device displays recommendation information according to the static balance estimated according to the motion of the foot of the user on the screen of the terminal device. For example, a video related to training for training a body part related to static balance is displayed on the screen of the terminal device as recommendation information according to the static balance estimated according to the motion of the foot of the user. According to the present aspect, the static balance estimated according to the feature of a gait of the user is displayed on the screen visually recognizable by the user, so that the user can confirm the information according to the static balance of the user.

### Second Example Embodiment

Next, a machine learning system according to a second example embodiment will be described with reference to the drawings. The machine learning system of the present example embodiment generates an estimation model for estimating the static balance according to the input of the feature amount by machine learning using the feature amount data extracted from the sensor data measured by the gait measurement device.

### (Configuration)

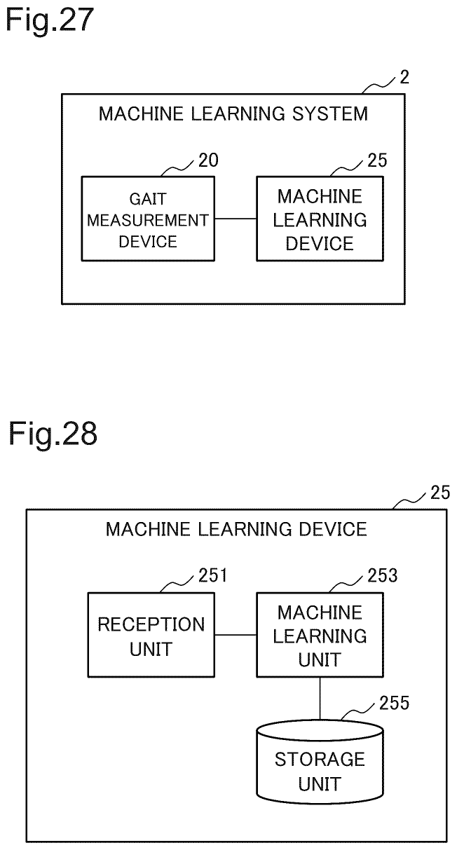

FIG. 27 is a block diagram illustrating an example of a configuration of a machine learning system 2 according to the present example embodiment. The machine learning system 2 includes a gait measurement device 20 and a machine learning device 25. The gait measurement device 20 and the machine learning device 25 may be connected by wire or wirelessly. The gait measurement device 20 and the machine learning device 25 may be configured by a single device. The machine learning system 2 may be configured only by the machine learning device 25 excluding the gait measurement device 20 from the configuration of the machine learning system 2. Although only one gait measurement device 20 is illustrated in FIG. 27, one (two in total) gait measurement device 20 may be disposed on each of the left and right feet. The machine learning device 25 may be configured not to be connected to the gait measurement device 20 but to perform machine learning using the feature amount data generated in advance by the gait measurement device 20 and stored in the database.

The gait measurement device 20 is installed at at least one of the left and right feet. The gait measurement device 20 has the same configuration as the gait measurement device 10 of the first example embodiment. The gait measurement device 20 includes an acceleration sensor and an angular velocity sensor. The gait measurement device 20 converts the measured physical quantity into digital data (also referred to as sensor data). The gait measurement device 20 generates normalized gait waveform data for one gait cycle from the time series data of the sensor data. The gait measurement device 20 generates feature amount data used for estimating the static balance. The gait measurement device 20 transmits the generated feature amount data to the machine learning device 25. The gait measurement device 20 may be configured to transmit the feature amount data to a database (not illustrated) accessed by the machine learning device 25. The feature amount data accumulated in the database is used for machine learning by the machine learning device 25.

The machine learning device 25 receives the feature amount data from the gait measurement device 20. When using the feature amount data accumulated in the database (not illustrated), the machine learning device 25 receives the feature amount data from the database. The machine learning device 25 performs machine learning using the received feature amount data. For example, the machine learning device 25 machine learns teacher data having feature amount data extracted from a plurality of pieces of subject gait waveform data as explanatory variables and a value related to static balance according to the feature amount data as an objective variable. The machine learning algorithm performed by the machine learning device 25 is not particularly limited. The machine learning device 25 generates an estimation model on which machine learning have been performed using teacher data related to a plurality of subjects. The machine learning device 25 stores the generated estimation model. The estimation model on which machine learning have been performed by the machine learning device 25 may be stored in a storage device outside the machine learning device 25.

### [Machine Learning Device]

Next, the operation of the machine learning device 25 will be described with reference to the drawings. FIG. 28 is a block diagram illustrating an example of a detailed configuration of the machine learning device 25. The machine learning device 25 includes a reception unit 251, a machine learning unit 253, and a storage unit 255.

The reception unit 251 receives the feature amount data from the gait measurement device 20. The reception unit 251 outputs the received feature amount data to the machine learning unit 253. The reception unit 251 may receive the feature amount data from the gait measurement device 20 via a wire such as a cable, or may receive the feature amount data from the gait measurement device 20 via wireless communication. For example, the reception unit 251 is configured to receive the feature amount data from the gait measurement device 20 via a wireless communication function (not illustrated) conforming to a standard such as Bluetooth (registered trademark) or Wi-Fi (registered trademark). The communication function of the reception unit 251 may conform to a standard other than Bluetooth (registered trademark) or Wi-Fi (registered trademark).

The machine learning unit 253 acquires the feature amount data from the reception unit 251. The machine learning unit 253 performs machine learning unit using the acquired feature amount data. For example, the machine learning unit 253 machine learns, as teacher data, a data set having the feature amount data extracted with respect to a gait of the subject as an explanatory variable and having the one leg standing position time of the subject an objective variable. For example, the machine learning unit 253 generates an estimation model that estimates the one leg standing position time according to the input of the feature amount data, the estimation model on which machine learning have been performed for a plurality of subjects. For example, the machine learning unit 253 generates an estimation model according to attribute data (age, height). For example, the machine learning unit 253 generates an estimation model that estimates the one leg standing position time as the static balance with the feature amount data extracted regarding a gait of the subject and the attribute data (age, height) of the subject as explanatory variables. The machine learning unit 253 stores an estimation model on which machine learning have been performed for a plurality of subjects in the storage unit 255.

For example, the machine learning unit 253 performs machine learning using a linear regression algorithm. For example, the machine learning unit 253 performs machine learning using an algorithm of a support vector machine (SVM). For example, the machine learning unit 253 performs machine learning using a Gaussian process regression (GPR) algorithm. For example, the machine learning unit 253 performs machine learning using an algorithm of random forest (RF). For example, the machine learning unit 253 may perform unsupervised machine learning unit that classifies a subject who is a generation source of the feature amount data according to the feature amount data. The machine learning algorithm performed by the machine learning unit 253 is not particularly limited.

The machine learning unit 253 may perform machine learning with the gait waveform data for one gait cycle as an explanatory variable. For example, the machine learning unit 253 executes supervised machine learning with the acceleration in the three axis direction, the angular velocity around the three axes, and the gait waveform data of the angle (posture angle) around the three axes as explanatory variables and the correct value of the static balance index as an objective variable. For example, in a case where the gait phase is set in increments of 1% in a 0 to 100% gait cycle, the machine learning unit 253 performs machine learning using 909 explanatory variables.

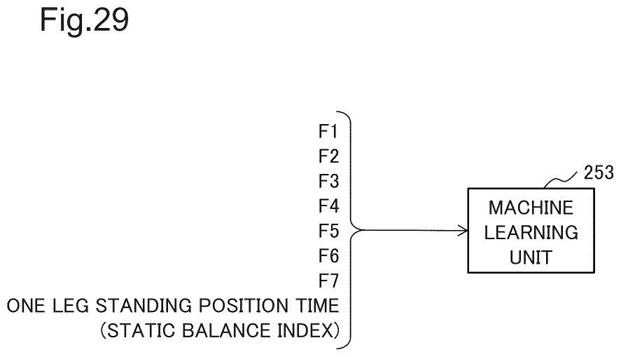

FIG. 29 is a conceptual diagram for describing machine learning for generating an estimation model. FIG. 29 is a conceptual diagram illustrating an example in which the machine learning unit 253 machine learns a data set of the feature amounts F1 to F7 that are explanatory variables and the one leg standing position time (static balance index) that is an objective variable as teacher data. For example, the machine learning unit 253 machine learns data related to a plurality of subjects, and generates an estimation model that outputs an output (estimation value) related to the one leg standing position time (static balance index) according to an input of a feature amount extracted from the sensor data.

The storage unit 255 stores the estimation model on which machine learning have been performed for a plurality of subjects. For example, the storage unit 255 stores an estimation model for estimating the static balance, the estimation model on which machine learning have been performed for a plurality of subjects. For example, the estimation model stored in the storage unit 255 is used for estimating the static balance by the static balance estimation device 13 of the first example embodiment.

As described above, the machine learning system of the present example embodiment includes the gait measurement device and the machine learning device. The gait measurement device acquires time series data of sensor data regarding motion of a foot. The gait measurement device extracts gait waveform data for one gait cycle from the time series data of the sensor data, and normalizes the extracted gait waveform data. The gait measurement device extracts, from a gait phase cluster configured by at least one temporally continuous gait phase, a feature amount used for estimating the static balance of the user from the normalized gait waveform data. The gait measurement device generates feature amount data including the extracted feature amount. The gait measurement device outputs the generated feature amount data to the machine learning device.

The machine learning device includes a reception unit, a machine learning unit, and a storage unit. The reception unit acquires the feature amount data generated by the gait measurement device. The machine learning unit performs machine learning unit using the feature amount data. The machine learning unit generates the estimation model that outputs the static balance according to the input of the feature amount (second feature amount) of the gait phase cluster extracted from the time series data of the sensor data measured along with a gait of the user. The estimation model generated by the machine learning unit is stored in the storage unit.

The machine learning system of the present example embodiment generates an estimation model by using the feature amount data measured by the gait measurement device. Therefore, according to the present aspect, it is possible to generate an estimation model capable of appropriately estimating the static balance in daily life without using an instrument for measuring the static balance.

### Third Example Embodiment

Next, a static balance estimation device according to a third example embodiment will be described with reference to the drawings. The static balance estimation device of the present example embodiment has a configuration in which the static balance estimation device included in the static balance estimation system of the first example embodiment is simplified.

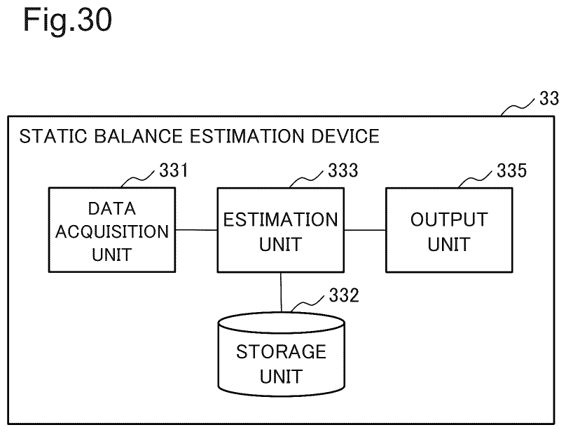

FIG. 30 is a block diagram illustrating an example of a configuration of a static balance estimation device 33 according to the present example embodiment. The static balance estimation device 33 includes a data acquisition unit 331, a storage unit 332, an estimation unit 333, and an output unit 335.

The data acquisition unit 331 acquires feature amount data including a feature amount extracted from the feature of a gait of the user and used for estimating the static balance index of the user. The storage unit 332 stores an estimation model that outputs the static balance index according to the input of the feature amount data. The estimation unit 333 inputs the acquired feature amount data to the estimation model, and estimates the static balance of the user according to the static balance index output from the estimation model. The output unit 335 outputs information related to the estimated static balance.

As described above, in the present example embodiment, the static balance of the user is estimated using the feature amount extracted from the feature of a gait of the user. Therefore, according to the present example embodiment, the static balance can be appropriately estimated in daily life without using an instrument for measuring the static balance.

### (Hardware)

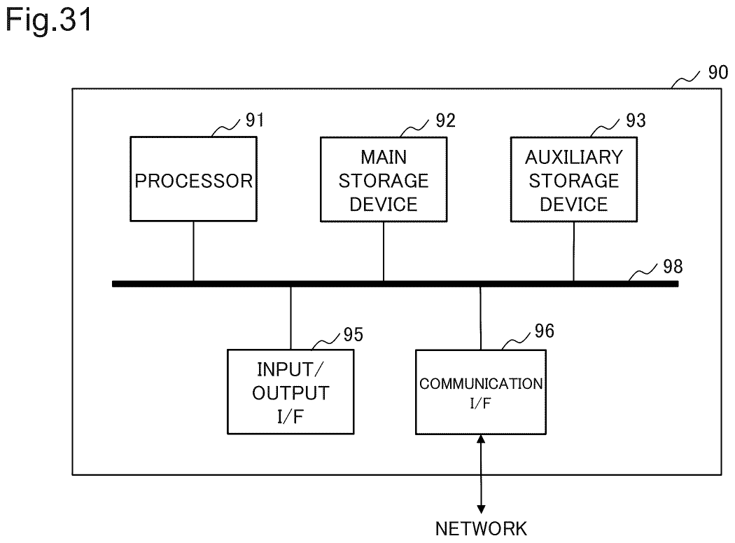

A hardware configuration for performing control and processing according to each example embodiment of the present disclosure will be described using an information processing device 90 of FIG. 31 as an example. The information processing device 90 in FIG. 31 is a configuration example for performing the process of the detection device of each example embodiment, and does not limit the scope of the present disclosure.

As illustrated in FIG. 31, the information processing device 90 includes a processor 91, a main storage device 92, an auxiliary storage device 93, an input/output interface 95, and a communication interface 96. In FIG. 31 the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops the program stored in the auxiliary storage device 93 or the like in the main storage device 92. The processor 91 executes the program developed in the main storage device 92. In the present example embodiment, a software program installed in the information processing device 90 may be used. The processor 91 executes control and processing according to each example embodiment.

The main storage device 92 has an area in which a program is developed. A program stored in the auxiliary storage device 93 or the like is developed in the main storage device 92 by the processor 91. The main storage device 92 is achieved by, for example, a volatile memory such as a dynamic random access memory (DRAM). A nonvolatile memory such as a magnetoresistive random access memory (MRAM) may be configured and added as the main storage device 92.

The auxiliary storage device 93 stores various pieces of data such as programs. The auxiliary storage device 93 is achieved by a local disk such as a hard disk or a flash memory. Various pieces of data may be stored in the main storage device 92, and the auxiliary storage device 93 may be omitted.

The input/output interface 95 is an interface that connects the information processing device 90 with a peripheral device based on a standard or a specification. The communication interface 96 is an interface that connects to an external system or a device through a network such as the Internet or an intranet in accordance with a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input of information and settings. In a case where the touch panel is used as the input device, the display screen of the display device may also serve as the interface of the input device. Data communication between the processor 91 and the input device may be mediated by the input/output interface 95.

The information processing device 90 may be provided with a display device that displays information. In a case where a display device is provided, the information processing device 90 preferably includes a display control device (not illustrated) that controls display of the display device. The display device may be connected to the information processing device 90 via the input/output interface 95.

The information processing device 90 may be provided with a drive device. The drive device mediates reading of data and a program from the recording medium, writing of a processing result of the information processing device 90 to the recording medium, and the like between the processor 91 and the recording medium (program recording medium). The drive device may be connected to the information processing device 90 via the input/output interface 95.

The above is an example of a hardware configuration for enabling control and processing according to each example embodiment of the present invention. The hardware configuration of FIG. 31 is an example of a hardware configuration for executing the arithmetic processing of the control unit according to each example embodiment, and does not limit the scope of the present invention. A program for causing a computer to execute control and processing according to each example embodiment is also included in the scope of the present invention. A program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a Universal Serial Bus (USB) memory or a secure digital (SD) card. The recording medium may be achieved by a magnetic recording medium such as a flexible disk, or another recording medium. In a case where the program executed by the processor is recorded in the recording medium, the recording medium corresponds to a program recording medium.

The components of each example embodiment may be combined in any manner. The components of each example embodiment may be achieved by software or may be achieved by a circuit.

While the present invention is described with reference to example embodiments thereof, the present invention is not limited to these example embodiments. Various modifications that can be understood by those of ordinary skill in the art can be made to the configuration and details of the present invention within the scope of the present invention.

Some or all of the above example embodiments may be described as the following Supplementary Notes, but are not limited to the following.

### (Supplementary Note 1)

A static balance estimation device including
a data acquisition unit that acquires feature amount data that include a feature amount to be used for estimating static balance of a user, the feature amount being extracted from a feature of a gait of the user,
a storage unit that stores an estimation model that outputs a static balance index related to input of the feature amount data,
an estimation unit that inputs the acquired feature amount data into the estimation model and estimates the static balance of the user according to the static balance index output by the estimation model, and
an output unit that outputs information related to the estimated static balance of the user.

### (Supplementary Note 2)

The static balance estimation device according to Supplementary Note 1, wherein
the data acquisition unit
acquires the feature amount data including a feature amount used to estimate a performance value of a one leg standing position test as the static balance index, the feature amount being extracted from gait waveform data generated using time series data of the sensor data related to motion of a foot.

### (Supplementary Note 3)

The static balance estimation device according to Supplementary Note 2, wherein
the storage unit
stores, with respect to a plurality of subjects, the estimation model generated by machine learning using teacher data having, as an explanatory variable, a feature amount used for estimating the static balance index, and having, as an objective variable, the static balance index of each of the plurality of subjects, and wherein
the estimation unit
inputs the feature amount data acquired regarding the user to the estimation model and estimates the static balance of the user according to the static balance index of the user, the static balance index being output from the estimation model.

### (Supplementary Note 4)

The static balance estimation device according to Supplementary Note 3, wherein
the storage unit
stores the estimation model on which machine learning have been performed using an explanatory variable including attribute data including at least one of an age and a height of each of the plurality of subjects, and wherein
the estimation unit
inputs the feature amount data and the attribute data related to the user to the estimation model, and estimates the static balance of the user according to the static balance index of the user, the static balance index being output from the estimation model.

### (Supplementary Note 5)

The static balance estimation device according to Supplementary Note 3 or 4, wherein
the storage unit
stores, with respect to the gait waveform data of the plurality of subjects, the estimation model generated by machine learning using teacher data having, as explanatory variables, a feature amount regarding an activity of a mesogluteus extracted from an end of a terminal swing period and a mid-stance period, a feature amount regarding an activity of each of a long adductor muscle and a sartorius extracted from a pre-swing period and an initial swing period, and a feature amount regarding an activity of an adductor/abductor group in a swing phase, and having, as an objective variable, the static balance index of each of the plurality of subjects, and wherein
the estimation unit
inputs the feature amount data acquired according to a gait of the user to the estimation model, and estimates the static balance of the user according to the static balance index of the user, the static balance index being output from the estimation model.

### (Supplementary Note 6)

The static balance estimation device according to Supplementary Note 5, wherein
the storage unit
stores, with respect to the plurality of subjects, the estimation model generated by machine learning using teacher data having, as explanatory variables, a feature amount, of a lateral acceleration, extracted from a mid-stance period of the gait waveform data, a feature amount, of a vertical acceleration, extracted from an end of a terminal swing period of the gait waveform data, a feature amount, of an angular velocity in a coronal plane, extracted from an initial swing period of the gait waveform data, a feature amount, of an angular velocity in a horizontal plane, extracted from a mid-stance period and a pre-swing period of the gait waveform data, and a feature amount related to a circumduction amount in a swing phase, and having, as an objective variable, the static balance index of each of the plurality of subjects, wherein
the data acquisition unit
acquires the feature amount data including a feature amount of a lateral acceleration in a mid-stance period of the gait waveform data, a feature amount of a vertical acceleration at an end of a terminal swing period of the gait waveform data, a feature amount of an angular velocity in a coronal plane in an initial swing period of the gait waveform data, a feature amount of an angular velocity in a horizontal plane in a mid-stance period and a pre-swing period of the gait waveform data, and a feature amount related to a circumduction amount in a swing phase that are extracted according to a gait of the user, and wherein
the estimation unit that
inputs the acquired feature amount data to the estimation model, and estimates the static balance of the user according to the static balance index of the user, the static balance index being output from the estimation model.

### (Supplementary Note 7)

The static balance estimation device according to Supplementary Note 6, wherein
the storage unit
stores, with respect to the plurality of subjects, the estimation model generated by machine learning using teacher data having an explanatory variable including a feature amount related to a foot angle at a timing of heel contact when a gait switches from a terminal swing period to a load response period of the gait waveform data of an angle in a horizontal plane and having, as an objective variable, the static balance index of each of the plurality of subjects, wherein
the data acquisition unit
acquires the feature amount data including a feature amount of a foot angle at a timing of heel contact when a gait switches from a terminal swing period to a load response period of the gait waveform data of an angle in a horizontal plane, and wherein
the estimation unit
inputs the acquired feature amount data to the estimation model, and estimates the static balance of the user according to the static balance index of the user, the static balance index being output from the estimation model.

### (Supplementary Note 8)

The static balance estimation device according to any one of Supplementary Notes 3 to 7, wherein
the estimation unit
estimates information related to the static balance of the user according to the static balance index estimated with respect to the user, and wherein
the output unit
outputs information related to the estimated static balance.

### (Supplementary Note 9)

A static balance estimation system including
the static balance estimation device according to any one of Supplementary Notes 1 to 8, and
a gait measurement device including a sensor that is installed at footwear of a user whose static balance is to be estimated, measures a spatial acceleration and a spatial angular velocity, generates sensor data related to motion of a foot using the measured spatial acceleration and the measured spatial angular velocity, and outputs the generated sensor data, and a feature amount data generation unit that acquires time series data of the sensor data including a feature of a gait, extracts gait waveform data for one gait cycle from the time series data of the sensor data, normalizes the extracted gait waveform data, extracts a feature amount used for estimating the static balance from the normalized gait waveform data from a gait phase cluster constituted by at least one temporally continuous gait phase, generates feature amount data including the extracted feature amount, and outputs the generated feature amount data to the static balance estimation device.

### (Supplementary Note 10)

The static balance estimation system according to Supplementary Note 9, wherein
the static balance estimation device
is mounted in a terminal device having a screen visible by the user, and
displays information related to the static balance estimated according to motion of a foot of the user on a screen of the terminal device.

### (Supplementary Note 11)

The static balance estimation system according to Supplementary Note 10, wherein
the static balance estimation device
displays recommendation information according to the static balance estimated according to motion of a foot of the user on a screen of the terminal device.

### (Supplementary Note 12)

The static balance estimation system according to Supplementary Note 11, wherein
the static balance estimation device
displays a video related to training for training a body part related to the static balance on a screen of the terminal device as the recommendation information according to the static balance estimated according to motion of a foot of the user.

### (Supplementary Note 13)

A method of estimating static balance including
a computer
acquiring feature amount data that include a feature amount to be used for estimating static balance of a user, the static balance being extracted from a feature of a gait of the user,
inputting the acquired feature amount data to an estimation model that outputs a static balance index according to input of the feature amount data,
estimating the static balance of the user according to the static balance index output from the estimation model, and
outputting information related to the estimated static balance of the user.
   (Supplementary Note 14) A program for causing a computer to execute the steps of
acquiring feature amount data that include a feature amount to be used for estimating static balance of a user, the static balance being extracted from a feature of a gait of the user,
inputting the acquired feature amount data to an estimation model that outputs a static balance index according to input of the feature amount data,
estimating the static balance of the user according to the static balance index output from the estimation model, and
outputting information related to the estimated static balance of the user.

### REFERENCE SIGNS LIST

1 static balance estimation system
2 machine learning system
10, 20 gait measurement device
11 sensor
12 feature amount data generation unit
13 static balance estimation device
25 machine learning device
111 acceleration sensor
112 angular velocity sensor
121 acquisition unit
122 normalization unit
123 extraction unit
125 generation unit
127 feature amount data output unit
131, 331 data acquisition unit
132, 332 storage unit
133, 333 estimation unit
135, 335 output unit
251 reception unit
253 machine learning unit
255 storage unit

What is claimed is:

1. A static balance estimation device comprising:
a storage configured to store an estimation model that outputs a static balance index corresponding to input of feature amount data used for estimating static balance;
a memory storing instructions; and
a processor connected to the memory and configured to execute the instructions to:
acquire feature amount data that include a feature amount to be used for estimating static balance of a user, the feature amount being extracted from a feature of a gait of the user;
input the acquired feature amount data into the estimation model to estimate the static balance of the user according to the static balance index output by the estimation model; and
output information related to the estimated static balance of the user.

2. The static balance estimation device according to claim 1, wherein
the processor is configured to execute the instructions to
acquire the feature amount data including a feature amount used to estimate a performance value of a one leg standing position test as the static balance index, the feature amount being extracted from gait waveform data generated using time series data of the sensor data related to motion of a foot.

3. The static balance estimation device according to claim 2, wherein
the storage stores, with respect to a plurality of subjects, the estimation model generated by machine learning using teacher data having, as an explanatory variable, a feature amount used for estimating the static balance index, and having, as an objective variable, the static balance index of each of the plurality of subjects, and
the processor is configured to execute the instructions to
input the feature amount data acquired regarding the user to the estimation model, and
estimate the static balance of the user according to the static balance index of the user, the static balance index being output from the estimation model.

4. The static balance estimation device according to claim 3, wherein
the storage stores the estimation model on which machine learning have been performed using an explanatory variable including attribute data including at least one of an age and a height of each of the plurality of subjects, and
the processor is configured to execute the instructions to
input the feature amount data and the attribute data related to the user to the estimation model, and
estimate the static balance of the user according to the static balance index of the user, the static balance index being output from the estimation model.

5. The static balance estimation device according to claim 3, wherein
the storage stores, with respect to the gait waveform data of the plurality of subjects, the estimation model generated by machine learning using teacher data having, as explanatory variables, a feature amount regarding an activity of a mesogluteus extracted from an end of a terminal swing period and a mid-stance period, a feature amount regarding an activity of each of a long adductor muscle and a sartorius extracted from a pre-swing period and an initial swing period, and a feature amount regarding an activity of an adductor/abductor group in a swing phase, and having, as an objective variable, the static balance index of each of the plurality of subjects, and
the processor is configured to execute the instructions to
input the feature amount data acquired according to a gait of the user to the estimation model, and
estimate the static balance of the user according to the static balance index of the user, the static balance index being output from the estimation model.

6. The static balance estimation device according to claim 5, wherein
the storage stores, with respect to the plurality of subjects, the estimation model generated by machine learning using teacher data having, as explanatory variables, a feature amount, of a lateral acceleration, extracted from a mid-stance period of the gait waveform data, a feature amount, of a vertical acceleration, extracted from an end of a terminal swing period of the gait waveform data, a feature amount, of an angular velocity in a coronal plane, extracted from an initial swing period of the gait waveform data, a feature amount, of an angular velocity in a horizontal plane, extracted from a mid-stance period and a pre-swing period of the gait waveform data, and a feature amount related to a circumduction amount in a swing phase, and having, as an objective variable, the static balance index of each of the plurality of subjects,
the processor is configured to execute the instructions to
acquire the feature amount data including a feature amount of a lateral acceleration in a mid-stance period of the gait waveform data, a feature amount of a vertical acceleration at an end of a terminal swing period of the gait waveform data, a feature amount of an angular velocity in a coronal plane in an initial swing period of the gait waveform data, a feature amount of an angular velocity in a horizontal plane in a mid-stance period and a pre-swing period of the gait waveform data, and a feature amount related to a circumduction amount in a swing phase that are extracted according to a gait of the user, and
input the acquired feature amount data to the estimation model, and
estimate the static balance of the user according to the static balance index of the user, the static balance index being output from the estimation model.

7. The static balance estimation device according to claim 6, wherein
the storage stores, with respect to the plurality of subjects, the estimation model generated by machine learning using teacher data having an explanatory variable including a feature amount related to a foot angle at a timing of heel contact when a gait switches from a terminal swing period to a load response period of the gait waveform data of an angle in a horizontal plane and having, as an objective variable, the static balance index of each of the plurality of subjects,
the processor is configured to execute the instructions to
acquire the feature amount data including a feature amount of a foot angle at a timing of heel contact when a gait switches from a terminal swing period to a load response period of the gait waveform data of an angle in a horizontal plane, and
input the acquired feature amount data to the estimation model, and
estimate the static balance of the user according to the static balance index of the user, the static balance index being output from the estimation model.

8. The static balance estimation device according to claim 3, wherein
the processor is configured to execute the instructions to
estimate information related to the static balance of the user according to the static balance index estimated with respect to the user, and
output information related to the estimated static balance.

9. A static balance estimation system comprising:
the static balance estimation device according to claim 1; and
a gait measurement device including
a sensor that is installed at footwear of a user whose static balance is to be estimated, measures a spatial acceleration and a spatial angular velocity, generates sensor data related to motion of a foot using the measured spatial acceleration and the measured spatial angular velocity, and outputs the generated sensor data, and
a memory storing instructions; and
a processor connected to the memory and configured to execute the instructions to
acquire time series data of the sensor data including a feature of a gait,
extract gait waveform data for one gait cycle from the time series data of the sensor data,
normalize the extracted gait waveform data,
extract, from a gait phase cluster constituted by at least one temporally continuous gait phase, a feature amount used for estimating the static balance from the normalized gait waveform data,
generate feature amount data including the extracted feature amount, and
output the generated feature amount data to the static balance estimation device.

10. The static balance estimation system according to claim 9, wherein
the static balance estimation device is mounted in a terminal device having a screen visible by the user, and
the processer of the static balance estimation device is configured to execute the instructions to display information related to the static balance estimated according to motion of a foot of the user on a screen of the terminal device.

11. The static balance estimation system according to claim 10, wherein
the processer of the static balance estimation device is configured to execute the instructions to
display recommendation information according to the static balance estimated according to motion of a foot of the user on a screen of the terminal device.

12. The static balance estimation system according to claim 11, wherein
the processer of the static balance estimation device is configured to execute the instructions to
display a video related to training for training a body part related to the static balance on a screen of the terminal device as the recommendation information according to the static balance estimated according to motion of a foot of the user.

13. A method of estimating static balance executed by a computer, the method comprising:
acquiring feature amount data that include a feature amount to be used for estimating static balance of a user, the static balance being extracted from a feature of a gait of the user;
inputting the acquired feature amount data to an estimation model that outputs a static balance index according to input of the feature amount data;
estimating the static balance of the user according to the static balance index output from the estimation model; and
outputting information related to the estimated static balance of the user.

14. A non-transitory recording medium recording a program for causing a computer to execute:
processing of acquiring feature amount data that include a feature amount to be used for estimating static balance of a user, the static balance being extracted from a feature of a gait of the user;
processing of inputting the acquired feature amount data to an estimation model that outputs a static balance index according to input of the feature amount data;
processing of estimating the static balance of the user according to the static balance index output from the estimation model; and
processing of outputting information related to the estimated static balance of the user.

15. The static balance estimation system according to claim 12, wherein
the processor of the mobility estimation device is configured to execute the instructions to
cause the recommendation information that supports the user for making decision about taking an action to be displayed on the screen of the terminal device.
